SEQUENCE LISTING (26 sequences)

SEQ ID NO: 1 - DNA, 2301 bp, Homo sapiens
  CDS (1)..(2301)
atggcggcgctgagcggtggcggtggtggcggcgcggagccgggccaggctctgttcaacggggacatggagcccgaggccggcgccggcgccggcgccgcggcctcttcggctgcggaccctgccattccggaggaggtgtggaatatcaaacaaatgattaagttgacacaggaacatatagaggccctattggacaaatttggtggggagcataatccaccatcaatatatctggaggcctatgaagaatacaccagcaagctagatgcactccaacaaagagaacaacagttattggaatctctggggaacggaactgatttttctgtttctagctctgcatcaatggataccgttacatcttcttcctcttctagcctttcagtgctaccttcatctctttcagtttttcaaaatcccacagatgtggcacggagcaaccccaagtcaccacaaaaacctatcgttagagtcttcctgcccaacaaacagaggacagtggtacctgcaaggtgtggagttacagtccgagacagtctaaagaaagcactgatgatgagaggtctaatcccagagtgctgtgctgtttacagaattcaggatggagagaagaaaccaattggttgggacactgatatttcctggcttactggagaagaattgcatgtggaagtgttggagaatgttccacttacaacacacaactttgtacgaaaaacgtttttcaccttagcattttgtgacttttgtcgaaagctgcttttccagggtttccgctgtcaaacatgtggttataaatttcaccagcgttgtagtacagaagttccactgatgtgtgttaattatgaccaacttgatttgctgtttgtctccaagttctttgaacaccacccaataccacaggaagaggcgtccttagcagagactgccctaacatctggatcatccccttccgcacccgcctcggactctattgggccccaaattctcaccagtccgtctccttcaaaatccattccaattccacagcccttccgaccagcagatgaagatcatcgaaatcaatttgggcaacgagaccgatcctcatcagctcccaatgtgcatataaacacaatagaacctgtcaatattgatgacttgattagagaccaaggatttcgtggtgatggaggatcaaccacaggtttgtctgctaccccccctgcctcattacctggctcactaactaacgtgaaagccttacagaaatctccaggacctcagcgagaaaggaagtcatcttcatcctcagaagacaggaatcgaatgaaaacacttggtagacgggactcgagtgatgattgggagattcctgatgggcagattacagtgggacaaagaattggatctggatcatttggaacagtctacaagggaaagtggcatggtgatgtggcagtgaaaatgttgaatgtgacagcacctacacctcagcagttacaagccttcaaaaatgaagtaggagtactcaggaaaacacgacatgtgaatatcctactcttcatgggctattccacaaagccacaactggctattgttacccagtggtgtgagggctccagcttgtatcaccatctccatatcattgagaccaaatttgagatgatcaaacttatagatattgcacgacagactgcacagggcatggattacttacacgccaagtcaatcatccacagagacctcaagagtaataatatatttcttcatgaagacctcacagtaaaaataggtgattttggtctagctacagtgaaatctcgatggagtgggtcccatcagtttgaacagttgtctggatccattttgtggatggcaccagaagtcatcagaatgcaagataaaaatccatacagctttcagtcagatgtatatgcatttggaattgttctgtatgaattgatgactggacagttaccttattcaaacatcaacaacagggaccagataatttttatggtgggacgaggatacctgtctccagatctcagtaaggtacggagtaactgtccaaaagccatgaagagattaatggcagagtgcctcaaaaagaaaagagatgagagaccactctttccccaaattctcgcctctattgagctgctggcccgctcattgccaaaaattcaccgcagtgcatcagaaccctccttgaatcgggctggtttccaaacagaggattttagtctatatgcttgtgcttctccaaaaacacccatccaggcagggggatatggtgcgtttcctgtccactga

SEQ ID NO: 2 - protein, 766 aa, Homo sapiens [sequence omitted]

SEQ ID NO: 3 - DNA, 42 bp, Artificial Sequence
  4825_BRAF_R primer
caggaaacagctatgaccttgaggactagttaacctggagga

SEQ ID NO: 4 - DNA, 44 bp, Artificial Sequence
  4826_BRAF_F primer
tgtaaaacgacggccagtagaatttttcttaaggggatctcttc

SEQ ID NO: 5 - DNA, 42 bp, Artificial Sequence
  4827_BRAF_R primer
caggaaacagctatgacccactgatttttgtgaatactggga

SEQ ID NO: 6 - DNA, 41 bp, Artificial Sequence
  4828_BRAF_F primer
tgtaaaacgacggccagtttaggaaagcatctcacctcatc

SEQ ID NO: 7 - DNA, 43 bp, Artificial Sequence
  4833_BRAF_R primer
caggaaacagctatgacctctttaaccacacaagtgttctttg

SEQ ID NO: 8 - DNA, 40 bp, Artificial Sequence
  4834_BRAF_F primer
tgtaaaacgacggccagtttttcccaagcatttatgacaa

SEQ ID NO: 9 - DNA, 45 bp, Artificial Sequence
  4835_BRAF_R primer
caggaaacagctatgaccacttaaaagaatgtggttaaagacaaa

SEQ ID NO: 10 - DNA, 38 bp, Artificial Sequence
  4836_BRAF_F primer
tgtaaaacgacggccagtcatggaacaaacaaggttgg

SEQ ID NO: 11 - DNA, 38 bp, Artificial Sequence
  4841_BRAF_R primer
caggaaacagctatgaccaggctgtggtatcctgctct

SEQ ID NO: 12 - DNA, 38 bp, Artificial Sequence
  4842_BRAF_F primer
tgtaaaacgacggccagtggcttgactggagtgaaagg

SEQ ID NO: 13 - DNA, 39 bp, Artificial Sequence
  4843_BRAF_R primer
caggaaacagctatgaccccaaaatttctaggtgtgcca

SEQ ID NO: 14 - DNA, 38 bp, Artificial Sequence
  4844_BRAF_F primer
tgtaaaacgacggccagtactccttttgtgggtttccc

SEQ ID NO: 15 - DNA, 39 bp, Artificial Sequence
  4845_BRAF_R primer
caggaaacagctatgacctgcgatggtcaagaaatatcc

SEQ ID NO: 16 - DNA, 41 bp, Artificial Sequence
  4846_BRAF_F primer
tgtaaaacgacggccagtatggtaaaagcattgctctagga

SEQ ID NO: 17 - DNA, 43 bp, Artificial Sequence
  21967_BRAF_F primer
tgtaaaacgacggccagtagctttttctgacaacattttaccg

SEQ ID NO: 18 - DNA, 41 bp, Artificial Sequence
  21968_BRAF_R primer
caggaaacagctatgacctgcaatccaaaagaatagcagcc

SEQ ID NO: 19 - DNA, 18 bp, Artificial Sequence
  -21M13 primer
tgtaaaacgacggccagt

SEQ ID NO: 20 - DNA, 18 bp, Artificial Sequence
  M13REV primer
caggaaacagctatgacc

SEQ ID NO: 21 - protein, 379 aa, Homo sapiens
MAAAAAQGGGGGEPRRTEGVGPGVPGEVEMVKGQPFDVGPRYTQLQYIGEGAYGMVSSAYDHVRKTRVAIKKISPFEHQTYCQRTLREIQILLRFRHENVIGIRDILRASTLEAMRDVYIVQDLMETDLYKLLKSQQLSNDHICYFLYQILRGLKYIHSANVLHRDLKPSNLLINTTCDLKICDFGLARIADPEHDHTGFLTEYVATRWYRAPEIMLNSKGYTKSIDIWSVGCILAEMLSNRPIFPGKHYLDQLNHILGILGSPSQEDLNCIINMKARNYLQSLPSKTKVAWAKLFPKSDSKALDLLDRMLTFNPNKRITVEEALAHPYLEQYYDPTDEPVAEEPFTFAMELDDLPKERLKELIFQETARFQPGVLEAP

SEQ ID NO: 22 - protein, 357 aa, Homo sapiens
MAAAAAQGGGGGEPRRTEGVGPGVPGEVEMVKGQPFDVGPRYTQLQYIGEGAYGMVSSAYDHVRKTRVAIKKISPFEHQTYCQRTLREIQILLRFRHENVIGIRDILRASTLEAMRDVYIVQDLMETDLYKLLKSQQLSNDHICYFLYQILRGLKYIHSANVLHRDLKPSNLLINTTCDLKICDFGLARIADPEHDHTGFLTEYVATRWYRAPEIMLNSKGYTKSIDIWSVGCILAEMLSNRPIFPGKHYLDQLNHILGILGSPSQEDLNCIINMKARNYLQSLPSKTKVAWAKLFPKSDSKALDLLDRMLTFNPNKRITVEEALAHPYLEQYYDPTDEVGQSPAAVGLGAGEQGGT

SEQ ID NO: 23 - protein, 360 aa, Homo sapiens [sequence omitted]

SEQ ID NO: 24 - protein, 360 aa, Homo sapiens [sequence omitted]

SEQ ID NO: 25 - protein, 393 aa, Homo sapiens [sequence omitted]

SEQ ID NO: 26 - protein, 400 aa, Homo sapiens [sequence omitted]

(19) United States
(12) Patent Application Publication
(10) Pub. No.: US 2011/0158944 A1
(43) Pub. Date: Jun. 30, 2011

(54) BRAF BIOMARKERS

(76) Inventors: Thomas J. Hosted, Summit, NJ (US); Jason S. Simon, Westfield, NJ (US); Marc M. Delorenzo, East Hanover, NJ (US); Donna Marie Carr, Kenilworth, NJ (US); William T. Windsor, East Brunswick, NJ (US); Ahmed A. Samatar, West Windsor, NJ (US)

(21) Appl. No.: 12/745,031

(22) PCT Filed: Nov. 26, 2008
(86) PCT No.: PCT/US08/84858

§ 371 Date: Jan. 7, 2011

(30) Foreign Application Priority Data
Nov. 30, 2007 (US) 60991351
Mar. 7, 2008 (US) 61034615

(51) Int. Cl. A61K 38/21; C12Q 1/68; A61P 35/00

(52) U.S. Cl. 424/85.7; 435/6.12

(57) ABSTRACT

The present invention provides, inter alia, methods for predicting the sensitivity of a disease, such as cancer, to an ERK1 or ERK2 or MEK inhibitor by detecting the presence of an allele of BRAF in cells mediating the disease. Methods of treatment are also provided.

This application claims the benefit of U.S. provisional patent application No. 60/991,351, filed Nov. 30, 2007 and U.S. provisional patent application No. 61/034,615, filed Mar. 7, 2008; each of which is herein incorporated by reference in its entirety.

### FIELD OF THE INVENTION

The field of the invention relates, generally, to methods for predicting sensitivity of a given disease to an ERK1 (Extracellular Signal-Regulated Kinases) or ERK2 inhibitor or MEK inhibitor as well as methods of treatment of such diseases.

### BACKGROUND OF THE INVENTION

The processes involved in tumor growth, progression, and metastasis are mediated by signaling pathways that are activated in cancer cells. The ERK pathway plays a central role in regulating mammalian cell growth by relaying extracellular signals from ligand-bound cell surface tyrosine kinase receptors such as members of the erbB family, PDGF, FGF, and VEGF receptor tyrosine kinase. Activation of the ERK pathway is via a cascade of phosphorylation events that begins with activation of Ras. Activation of Ras leads to the recruitment and activation of Raf, a serine-threonine kinase. The RAF component of this pathway is a serine/threonine kinase and has three isoforms (BRAF, ARAF, and RAF1) that activate the MEK-ERK cascade. Activated Raf then phosphorylates and activates MEK1/2, which then phosphorylates and activates ERK1/2. When activated, ERK1/2 phosphorylates several downstream targets involved in a multitude of cellular events including cytoskeletal changes and transcriptional activation. The ERK/MAPK pathway is one of the most important for cell proliferation, and it is believed that the ERK/MAPK pathway is frequently activated in many tumors. Ras genes, which are upstream of ERK1/2, are mutated in several cancers including colorectal, melanoma, breast and pancreatic tumors. The high Ras activity is accompanied by elevated ERK activity in many human tumors. In addition, mutations of BRAF, a serine-threonine kinase of the Raf family, are associated with increased kinase activity. These observations indicate that the ERK1/2 signaling pathway is an attractive pathway for anticancer therapies in a broad spectrum of human tumors.

Some inhibitors of ERK1 and ERK2 are known (see e.g., WO2007/70398) and have, indeed, demonstrated to be effective anti-cancer agents. Factors including, e.g., individual genetic variability, can, however, render a particular patient non-responsive to a given therapy. The use of biomarkers for responsiveness to a given therapy is, thus, a useful tool for quickly and conveniently determining the responsiveness of a patient before a course of treatment is initiated. Often, early, successful treatment of a given cancer is critical to the patient's clinical outcome. The use of biomarkers can aid in this process by quickly helping to identify treatments likely to be effective in a given patient and/or helping to eliminate treatments likely to be ineffective in a given patient. Another benefit of the use of biomarkers relates to patient compliance. Patients assured that a given inhibitor therapy will likely be effective against their specific tumor will exhibit an enhanced likelihood of continuing with the prescribed inhibitor-based regimen over time. There is a need in the art for biomarkers for predicting cancer sensitivity to an ERK1 or ERK2 or MEK inhibitor-based cancer therapy.

### SUMMARY OF THE INVENTION

The present invention addresses this need in the art by providing a BRAF genetic biomarker which is effective at predicting sensitivity of a cancer cell's spread, growth or survival to an ERK1 or ERK2 or MEK (e.g., MEK1 or MEK2) inhibitor.

The present invention provides a method for evaluating sensitivity of malignant or neoplastic cells to an ERK1 or ERK2 or MEK inhibitor comprising determining if said cells are characterized by a homozygous or heterozygous V600E BRAF genotype or a homozygous or heterozygous V600D BRAF genotype or any BRAF genotype characterized by a gain-of-function phenotype; wherein said cells are determined to be sensitive if said genotype is detected. In an embodiment of the invention, said malignant or neoplastic cells mediate a medical condition selected from the group consisting of gastric cancer, any renal cancer, rhabdomyosarcoma, cholangiocarcinoma, lung cancer, pancreatic cancer, colon cancer, myeloid leukemias, thyroid cancer, myelodysplastic syndrome, bladder carcinoma, epidermal carcinoma, melanoma, breast cancer, prostate cancer, head and neck cancers, ovarian cancer, brain cancers, cancers of mesenchymal origin, sarcomas, tetracarcinomas, neuroblastomas, kidney carcinomas, hepatomas, non-Hodgkin's lymphoma, multiple myeloma, and anaplastic thyroid carcinoma. In an embodiment of the invention, said ERK or MEK inhibitor is represented by a structural formula selected from the group consisting of:

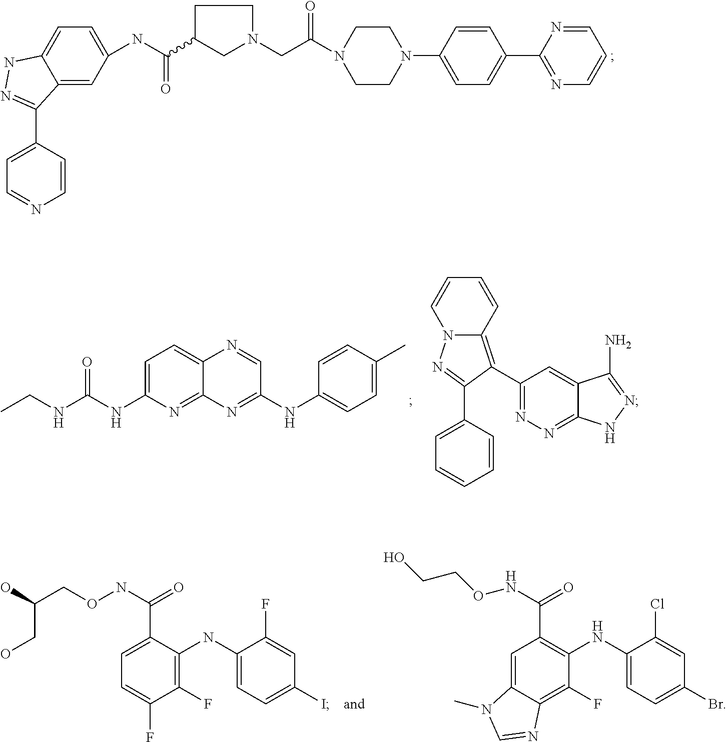

In an embodiment of the invention, cells characterized by a homozygous V600E BRAF genotype are determined to be sensitive. In an embodiment of the invention, the cells are obtained from an in vitro or in vivo source. For example, in an embodiment of the invention, the method comprises (a) obtaining a sample of one or more malignant or neoplastic cells from the body of a subject; (b) determining if said malignant or neoplastic cells are characterized by a homozygous or heterozygous V600E BRAF genotype or a heterozygous V600D BRAF genotype or any BRAF genotype characterized by a gain-of-function phenotype; wherein the cells are determined to be sensitive to said inhibitor is said genotype is detected in said cells. In an embodiment of the invention, the method further comprises treating the malignant or neoplastic cells in the subject by administering a therapeutically effective amount of the inhibitor, optionally in association with a therapeutically effective amount of a further chemotherapeutic agent (e.g., temozolomide or calcitriol), to said subject, if said cells are determined to be sensitive.

The present invention also provides a method for selecting a subject with malignant or neoplastic cells for treatment with an ERK1 or ERK2 or MEK inhibitor comprising evaluating sensitivity of the malignant or neoplastic cells to said inhibitor by the methods discussed herein; wherein said subject is selected if said cells are determined to be sensitive. In an embodiment of the invention, said malignant or neoplastic cells mediate a medical condition selected from the group consisting of gastric cancer, any renal cancer, rhabdomyosarcoma, cholangiocarcinoma, lung cancer, pancreatic cancer, colon cancer, myeloid leukemias, thyroid cancer, myelodysplastic syndrome, bladder carcinoma, epidermal carcinoma, melanoma, breast cancer, prostate cancer, head and neck cancers, ovarian cancer, brain cancers, cancers of mesenchymal origin, sarcomas, tetracarcinomas, neuroblastomas, kidney carcinomas, hepatomas, non-Hodgkin's lymphoma, multiple myeloma, and anaplastic thyroid carcinoma. In an embodiment of the invention, the method further comprises treating the malignant or neoplastic cells in the subject by administering a therapeutically effective amount of the inhibitor, optionally in association with a therapeutically effective amount of a further chemotherapeutic agent (e.g., temozolomide or calcitriol), to said subject, if said subject is selected.

The present invention provides a method for identifying a subject with malignant or neoplastic cells sensitive to an ERK1 or ERK2 or MEK inhibitor comprising evaluating sensitivity of the malignant or neoplastic cells to said inhibitor by methods discussed herein; wherein said subject is identified if said cells are determined to be sensitive. In an embodiment of the invention, the malignant or neoplastic cells mediate a medical condition selected from the group consisting of gastric cancer, any renal cancer, rhabdomyosarcoma, cholangiocarcinoma, lung cancer, pancreatic cancer, colon cancer, myeloid leukemias, thyroid cancer, myelodysplastic syndrome, bladder carcinoma, epidermal carcinoma, melanoma, breast cancer, prostate cancer, head and neck cancers, ovarian cancer, brain cancers, cancers of mesenchymal origin, sarcomas, tetracarcinomas, neuroblastomas, kidney carcinomas, hepatomas, non-Hodgkin's lymphoma, multiple myeloma, and anaplastic thyroid carcinoma. In an embodiment of the invention, the method further comprises treating the malignant or neoplastic cells in the subject by administering a therapeutically effective amount of the inhibitor, optionally in association with a therapeutically effective amount of a further chemotherapeutic agent (e.g., temozolomide or calcitriol), to said subject if the subject is identified.

The present invention further provides a method for treating a medical condition mediated by malignant or neoplastic cells with an ERK1 or ERK2 or MEK inhibitor comprising evaluating sensitivity of the malignant or neoplastic cells to said inhibitor by methods set forth herein and, if said cells are determined to be sensitive, continuing or commencing treatment by administering, to the subject, a therapeutically effective dose of the inhibitor. In an embodiment of the invention, the medical condition is selected from the group consisting of gastric cancer, any renal cancer, rhabdomyosarcoma, cholangiocarcinoma, lung cancer, pancreatic cancer, colon cancer, myeloid leukemias, thyroid cancer, myelodysplastic syndrome, bladder carcinoma, epidermal carcinoma, melanoma, breast cancer, prostate cancer, head and neck cancers, ovarian cancer, brain cancers, cancers of mesenchymal origin, sarcomas, tetracarcinomas, neuroblastomas, kidney carcinomas, hepatomas, non-Hodgkin's lymphoma, multiple myeloma, and anaplastic thyroid carcinoma. For example, in an embodiment of the invention, said malignant or neoplastic cells are in a tumor or mediate a non-solid cancer.

The scope of the present invention also encompasses a method for selecting a therapy for a patient having a medical condition mediated by malignant or neoplastic cells comprising evaluating sensitivity of the cells to an ERK1 or ERK2 or MEK inhibitor by methods discussed herein; wherein said inhibitor is selected as the therapy if said cells are determined to be sensitive to the inhibitor. In an embodiment of the invention, the medical condition is selected from the group consisting of gastric cancer, any renal cancer, rhabdomyosarcoma, cholangiocarcinoma, lung cancer, pancreatic cancer, colon cancer, myeloid leukemias, thyroid cancer, myelodysplastic syndrome, bladder carcinoma, epidermal carcinoma, melanoma, breast cancer, prostate cancer, head and neck cancers, ovarian cancer, brain cancers, cancers of mesenchymal origin, sarcomas, tetracarcinomas, neuroblastomas, kidney carcinomas, hepatomas, non-Hodgkin's lymphoma, multiple myeloma, and anaplastic thyroid carcinoma. In an embodiment of the invention, the method further comprises treating the medical condition in the subject by administering a therapeutically effective amount of the inhibitor, optionally in association with a therapeutically effective amount of a further chemotherapeutic agent (e.g., temozolomide or calcitriol), to said subject, if said therapy is selected.

The scope of the present invention also includes a method for selecting a dose of an ERK1 or ERK2 or MEK inhibitor to be administered, to a subject, having a medical condition mediated by malignant or neoplastic cells, comprising evaluating sensitivity of the cells by method set forth herein; wherein a lower dose is selected if said cells are determined to be sensitive relative to a dose selected if said cells are not determined to be sensitive. In an embodiment of the invention, the medical condition is selected from the group consisting of gastric cancer, any renal cancer, rhabdomyosarcoma, cholangiocarcinoma, lung cancer, pancreatic cancer, colon cancer, myeloid leukemias, thyroid cancer, myelodysplastic syndrome, bladder carcinoma, epidermal carcinoma, melanoma, breast cancer, prostate cancer, head and neck cancers, ovarian cancer, brain cancers, cancers of mesenchymal origin, sarcomas, tetracarcinomas, neuroblastomas, kidney carcinomas, hepatomas, non-Hodgkin's lymphoma, multiple myeloma, and anaplastic thyroid carcinoma. In an embodiment of the invention, the method further comprises administering the selected dose of the inhibitor, optionally in association with a therapeutically effective amount of a further chemotherapeutic agent (e.g., temozolomide or calcitriol), to said subject.

### DETAILED DESCRIPTION OF THE INVENTION

The BRAF genotype status of a cell, e.g., a cell line (homozygous V600E BRAF or heterozygous V600E BRAF or homozygous V600D BRAF or heterozygous V600D BRAF or any BRAF genotype characterized by a gain-of-function phenotype) is a novel predictive biomarker for compound sensitivity (e.g., of cells in a tumor in a patient) to ERK1/2 or MEK kinase inhibitors. An important feature and advantage of the present invention is that the V600 BRAF mutation genotype status may be used as a predictive biomarker for sensitivity to additional ERK1/2 or MEK inhibitor compounds and, thus, aid in the development of novel chemotherapeutics for human cancer including melanoma. The correlation of ERK1/2 or MEK inhibitor drug response to the mutational status of BRAF, a recognized oncogene, will allow diagnostic tests to be developed for BRAF to predict ERK1/2 and MEK compound sensitivity profiles in human tumor tissues, cell lines and mouse xenograft models.

In an embodiment of the invention, a cell is generally considered more sensitive to an ERK1 or ERK2 inhibitor if growth inhibition is characterized by an 1050 value of about 100 nM or lower. An IC50 value of over 100 nM is generally considered resistant (i.e., less sensitive).

A cell is generally considered more sensitive to a MEK inhibitor is its growth inhibition is characterized by an IC50 value of about 10 nM or lower. An IC50 value of over 10 nM is generally considered resistant (i.e., less sensitive).

Generally, the MEK inhibitor or ERK inhibitor (e.g., as set forth herein) sensitivity associated with a BRAF V600 mutant genotype as set forth herein is ranked as follows: homozygote>heterozygote>wild-type.

A subject includes any organism, including, e.g., an animal, such as a mammal (e.g., a human).

Neoplastic cells exhibit abnormally high levels of proliferation and may form a tumor or mass. Neoplasms may be benign or malignant. In general, malignant cells and tumors can invade and destroy nearby tissue and organs and spread to other parts of the body.

The present invention provides methods for treating malignant or neoplastic cells or a medical condition. Such methods include embodiments wherein growth, survival or spread (e.g., metastasis) of such cells are inhibited to any degree.

The scope of the present invention also includes embodiments wherein solid tumor diseases and non-solid tumor diseases are treated. Non-solid tumor diseases include embodiments wherein the disease is not mediated by a solid tumor or mass, for example, blood cancers such as leukemia.

The term “gain-of-function” with respect to BRAF would be understood by any practitioner of ordinary skill in the art (see e.g., Hoeflich et al., Cancer Res. (2006) 66(2): 999-1006). For example, in an embodiment of the invention a gain-of-function BRAF genotype promotes increased or constitutive BRAF-mediated intracellular signaling which may lead, e.g., to increased cellular proliferation and/or a transformed phenotype—e.g., by increased or constitutive phosphorylation of MEK (e.g., MEK1 or MEK2).

### BRAF

The term BRAF includes any human BRAF gene or protein whatsoever. BRAF is known by several names including, for example, B-Raf proto-oncogene serine/threonine-protein kinase, 94 kDa B-raf protein and v-Raf murine sarcoma viral oncogene homolog B1. A V600E or V600D mutant of BRAF comprises a glutamic acid or aspartic acid in place of valine at position 600. The present invention includes such mutant BRAF polypeptides and polynucleotides as well as uses thereof (e.g., as set forth herein).

In an embodiment of the invention, BRAF comprises the following amino acid sequence:

(SEQ ID NO: 2)
MAALSGGGGG GAEPGQALFN GDMEPEAGAG AGAAASSAAD
PAIPEEVWNI KQMIKLTQEH IEALLDKFGG EHNPPSIYLE
AYEEYTSKLD ALQQREQQLL ESLGNGTDFS VSSSASMDTV
TSSSSSSLSV LPSSLSVFQN PTDVARSNPK SPQKPIVRVF
LPNKQRTVVP ARCGVTVRDS LKKALMMRGL IPECCAVYRI
QDGEKKPIGW DTDISWLTGE ELHVEVLENV PLTTHNFVRK
TFFTLAFCDF CRKLLFQGFR CQTCGYKFHQ RCSTEVPLMC
VNYDQLDLLF VSKFFEHHPI PQEEASLAET ALTSGSSPSA
PASDSIGPQI LTSPSPSKSI PIPQPFRPAD EDHRNQFGQR
DRSSSAPNVH INTIEPVNID DLIRDQGFRG DGGSTTGLSA
TPPASLPGSL TNVKALQKSP GPQRERKSSS SSEDRNRMKT
LGRRDSSDDW EIPDGQITVG QRIGSGSFGT VYKGKWHGDV
AVKMLNVTAP TPQQLQAFKN EVGVLRKTRH VNILLFMGYS
TKPQLAIVTQ WCEGSSLYHH LHIIETKFEM IKLIDIARQT
AQGMDYLHAK SIIHRDLKSN NIFLHEDLTV KIGDFGLATV
KSRWSGSHQF EQLSGSILWM APEVIRMQDK NPYSFQSDVY
AFGIVLYELM TGQLPYSNIN NRDQIIFMVG RGYLSPDLSK
VRSNCPKAMK RLMAECLKKK RDERPLFPQI LASIELLARS
LPKIHRSASE PSLNRAGFQT EDFSLYACAS PKTPIQAGGY
GAFPVH

Valine 600, which is mutated to a glutamic acid in a V600E mutant or to an aspartic acid in a V600D mutant, is underlined and in bold faced font.

In an embodiment of the invention, BRAF, is encoded by the following polynucleotide:

(SEQ ID NO: 1)
ATGGCGGCGCTGAGCGGTGGCGGTGGTGGCGGCGCGGAGCCGGGCCAGGCTCTGTTCAACGGGGACATGG
AGCCCGAGGCCGGCGCCGGCGCCGGCGCCGCGGCCTCTTCGGCTGCGGACCCTGCCATTCCGGAGGAGGT
GTGGAATATCAAACAAATGATTAAGTTGACACAGGAACATATAGAGGCCCTATTGGACAAATTTGGTGGG
GAGCATAATCCACCATCAATATATCTGGAGGCCTATGAAGAATACACCAGCAAGCTAGATGCACTCCAAC
AAAGAGAACAACAGTTATTGGAATCTCTGGGGAACGGAACTGATTTTTCTGTTTCTAGCTCTGCATCAAT
GGATACCGTTACATCTTCTTCCTCTTCTAGCCTTTCAGTGCTACCTTCATCTCTTTCAGTTTTTCAAAAT
CCCACAGATGTGGCACGGAGCAACCCCAAGTCACCACAAAAACCTATCGTTAGAGTCTTCCTGCCCAACA
AACAGAGGACAGTGGTACCTGCAAGGTGTGGAGTTACAGTCCGAGACAGTCTAAAGAAAGCACTGATGAT
GAGAGGTCTAATCCCAGAGTGCTGTGCTGTTTACAGAATTCAGGATGGAGAGAAGAAACCAATTGGTTGG
GACACTGATATTTCCTGGCTTACTGGAGAAGAATTGCATGTGGAAGTGTTGGAGAATGTTCCACTTACAA
CACACAACTTTGTACGAAAAACGTTTTTCACCTTAGCATTTTGTGACTTTTGTCGAAAGCTGCTTTTCCA
GGGTTTCCGCTGTCAAACATGTGGTTATAAATTTCACCAGCGTTGTAGTACAGAAGTTCCACTGATGTGT
GTTAATTATGACCAACTTGATTTGCTGTTTGTCTCCAAGTTCTTTGAACACCACCCAATACCACAGGAAG
AGGCGTCCTTAGCAGAGACTGCCCTAACATCTGGATCATCCCCTTCCGCACCCGCCTCGGACTCTATTGG
GCCCCAAATTCTCACCAGTCCGTCTCCTTCAAAATCCATTCCAATTCCACAGCCCTTCCGACCAGCAGAT
GAAGATCATCGAAATCAATTTGGGCAACGAGACCGATCCTCATCAGCTCCCAATGTGCATATAAACACAA
TAGAACCTGTCAATATTGATGACTTGATTAGAGACCAAGGATTTCGTGGTGATGGAGGATCAACCACAGG
TTTGTCTGCTACCCCCCCTGCCTCATTACCTGGCTCACTAACTAACGTGAAAGCCTTACAGAAATCTCCA
GGACCTCAGCGAGAAAGGAAGTCATCTTCATCCTCAGAAGACAGGAATCGAATGAAAACACTTGGTAGAC
GGGACTCGAGTGATGATTGGGAGATTCCTGATGGGCAGATTACAGTGGGACAAAGAATTGGATCTGGATC
ATTTGGAACAGTCTACAAGGGAAAGTGGCATGGTGATGTGGCAGTGAAAATGTTGAATGTGACAGCACCT
ACACCTCAGCAGTTACAAGCCTTCAAAAATGAAGTAGGAGTACTCAGGAAAACACGACATGTGAATATCC
TACTCTTCATGGGCTATTCCACAAAGCCACAACTGGCTATTGTTACCCAGTGGTGTGAGGGCTCCAGCTT
GTATCACCATCTCCATATCATTGAGACCAAATTTGAGATGATCAAACTTATAGATATTGCACGACAGACT
GCACAGGGCATGGATTACTTACACGCCAAGTCAATCATCCACAGAGACCTCAAGAGTAATAATATATTTC
TTCATGAAGACCTCACAGTAAAAATAGGTGATTTTGGTCTAGCTACAGTGAAATCTCGATGGAGTGGGTC
CCATCAGTTTGAACAGTTGTCTGGATCCATTTTGTGGATGGCACCAGAAGTCATCAGAATGCAAGATAAA
AATCCATACAGCTTTCAGTCAGATGTATATGCATTTGGAATTGTTCTGTATGAATTGATGACTGGACAGT
TACCTTATTCAAACATCAACAACAGGGACCAGATAATTTTTATGGTGGGACGAGGATACCTGTCTCCAGA
TCTCAGTAAGGTACGGAGTAACTGTCCAAAAGCCATGAAGAGATTAATGGCAGAGTGCCTCAAAAAGAAA
AGAGATGAGAGACCACTCTTTCCCCAAATTCTCGCCTCTATTGAGCTGCTGGCCCGCTCATTGCCAAAAA
TTCACCGCAGTGCATCAGAACCCTCCTTGAATCGGGCTGGTTTCCAAACAGAGGATTTTAGTCTATATGC
TTGTGCTTCTCCAAAAACACCCATCCAGGCAGGGGGATATGGTGCGTTTCCTGTCCACTGA

The location of an allele, such as a V600E or V600D mutation, in BRAF may be identified by its location in a consensus or reference sequence relative to the initiation codon (ATG) for protein translation. The skilled artisan understands that the location of a particular allele may not occur at precisely the same position in a reference or context sequence in each individual in a population of interest due to the presence of one or more insertions or deletions in that individual as compared to the consensus or reference sequence. Thus, the skilled artisan will understand that specifying the location of any allele described herein by reference to a particular position in a reference or context sequence (or with respect to an initiation codon in such a sequence) is merely for convenience and that any specifically enumerated nucleotide position literally includes whatever nucleotide position the same allele is actually located at in the same locus in any individual being tested for the presence or absence of a biomarker of the invention using any of the genotyping methods described herein or other genotyping methods well-known in the art.

The codon encoding valine 600, which is mutated to a glutamic acid in a V600E mutant or to an aspartic acid in a V600D mutant, is underlined and in bold faced font. The present invention includes embodiments wherein the codon is any codon with encodes valine (e.g., GTT, GTC, GTA or GTG) or, in a mutant allele, wherein the codon encoding glutamic acid is any such codon (e.g., GAA or GAG) or wherein the codon encoding aspartic acid is any such codon (e.g., GAT or GAC).

In an embodiment of the invention, a V600D mutation is caused by a tandem mutation, TG1796-97AT.

### ERK and MEK

The extracellular signal-regulated kinases 1 and 2 (ERK1 and ERK2), also called p44 and p42 MAP kinases, are members of the Mitogen Activated Protein Kinase (MAPK) family of proteins found in eukaryotes. Because the 44 kDa ERK1 and the 42 kDa ERK2 are highly homologous and both function in the same protein kinase cascade, the two proteins are often referred to collectively as ERK1/2 or p44/p42 MAP kinase. The ERK1/2 signaling cascade has been shown to be a critical regulator of cell differentiation, cell physiology and neuronal function. Aberrant control of ERK1/2 activity has been implicated in a variety of pathological conditions, including cancer and autoimmune diseases.

The terms “ERK1/2” and “ERK” and the like refer to ERK 1 and ERK2. The terms “MEK” and “MEK1/2” and the like refer to MEK1 and MEK2.

The official name of ERK1 is mitogen-activated protein kinase 3. In an embodiment of the invention, human ERK1 comprises the following amino acid sequence:

(SEQ ID NO: 21)
MAAAAAQGGGGGEPRRTEGVGPGVPGEVEMVKGQPFDVGPRYTQLQYIG
EGAYGMVSSAYDHVRKTRVAIKKISPFEHQTYCQRTLREIQILLRFRHE
NVIGIRDILRASTLEAMRDVYIVQDLMETDLYKLLKSQQLSNDHICYFL
YQILRGLKYIHSANVLHRDLKPSNLLINTTCDLKICDFGLARIADPEHD
HTGFLTEYVATRWYRAPEIMLNSKGYTKSIDIWSVGCILAEMLSNRPIF
PGKHYLDQLNHILGILGSPSQEDLNCIINMKARNYLQSLPSKTKVAWAK
LEPKSDSKALDLLDRMLTENPNKRITVEEALAHPYLEQYYDPTDEPVAE
EPFTFAMELDDLPKERLKELIFQETARFQPGVLEAP

or the following amino acid sequence:

(SEQ ID NO: 22)
MAAAAAQGGGGGEPRRTEGVGPGVPGEVEMVKGQPFDVGPRYTQLQYIG
EGAYGMVSSAYDHVRKTRVAIKKISPFEHQTYCQRTLREIQILLRFRHE
NVIGIRDILRASTLEAMRDVYIVQDLMETDLYKLLKSQQLSNDHICYFL
YQILRGLKYIHSANVLHRDLKPSNLLINTTCDLKICDFGLARIADPEHD
HTGFLTEYVATRWYRAPEIMLNSKGYTKSIDIWSVGCILAEMLSNRPIF
PGKHYLDQLNHILGILGSPSQEDLNCIINMKARNYLQSLPSKTKVAWAK
LFPKSDSKALDLLDRMLTFNPNKRITVEEALAHPYLEQYYDPTDEVGQS
PAAVGLGAGEQGGT

In an embodiment of the invention, human ERK2 which is also known as mitogen-activated protein kinase 1; p40; p41; ERT1; MAPK2; PRKM1; PRKM2; P42MAPK; or p41mapk, comprises the following amino acid sequence:

(SEQ ID NO: 23)
MAAAAAAGAGPEMVRGQVFDVGPRYTNLSYIGEGAYGMVCSAYDNVNKV
RVAIKKISPFEHQTYCQRTLREIKILLRFRHENIIGINDIIRAPTIEQM
KDVYIVQDLMETDLYKLLKTQHLSNDHICYFLYQILRGLKYIHSANVLH
RDLKPSNLLLNTTCDLKICDFGLARVADPDHDHTGFLTEYVATRWYRAP
EIMLNSKGYTKSIDIWSVGCILAEMLSNRPIFPGKHYLDQLNHILGILG
SPSQEDLNCIINLKARNYLLSLPHKNKVPWNRLFPNADSKALDLLDKML
TFNPHKRIEVEQALAHPYLEQYYDPSDEPIAEAPFKFDMELDDLPKEKL
KELIFEETARFQPGYRS

or the following amino acid sequence:

(SEQ ID NO: 24)
MAAAAAAGAGPEMVRGQVFDVGPRYTNLSYIGEGAYGMVCSAYDNVNKV
RVAIKKISPFEHQTYCQRTLREIKILLRFRHENIIGINDIIRAPTIEQM
KDVYIVQDLMETDLYKLLKTQHLSNDHICYFLYQILRGLKYIHSANVLH
RDLKPSNLLLNTTCDLKICDFGLARVADPDHDHTGFLTEYVATRWYRAP
EIMLNSKGYTKSIDIWSVGCILAEMLSNRPIFPGKHYLDQLNHILGILG
SPSQEDLNCIINLKARNYLLSLPHKNKVPWNRLFPNADSKALDLLDKML
TFNPHKRIEVEQALAHPYLEQYYDPSDEPIAEAPFKFDMELDDLPKEKL
KELIFEETARFQPGYRS

In an embodiment of the invention, human MEK1, which is also known as MAP2K1, MKK1; MAPKK1; PRKMK1, comprises the following amino acid sequence:

(SEQ ID NO: 25)
MPKKKPTPIQLNPAPDGSAVNGTSSAETNLEALQKKLEELELDEQQRKR
LEAFLTQKQKVGELKDDDFEKISELGAGNGGVVFKVSHKPSGLVMARKL
IHLEIKPAIRNQIIRELQVLHECNSPYIVGFYGAFYSDGEISICMEHMD
GGSLDQVLKKAGRIPEQILGKVSIAVIKGLTYLREKHKIMHRDVKPSNI
LVNSRGEIKLCDFGVSGQLIDSMANSFVGTRSYMSPERLQGTHYSVQSD
IWSMGLSLVEMAVGRYPIPPPDAKELELMFGCQVEGDAAETPPRPRTPG
RPLSSYGMDSRPPMAIFELLDYIVNEPPPKLPSGVFSLEFQDFVNKCLI
KNPAERADLKQLMVHAFIKRSDAEEVDFAGWLCSTIGLNQPSTPTHAAG
V

In an embodiment of the invention, human MEK2, which is also known as mitogen-activated protein kinase kinase 2, MAP2K2; MAPKK2, MKK2, PRKMK2 comprises the following amino acid sequence. The protein encoded by this gene is a dual specificity protein kinase that belongs to the MAP kinase kinase family. This kinase is known to play a critical role in mitogen growth factor signal transduction. It phosphorylates and thus activates MAPK1/ERK2.

(SEQ ID NO: 26)
MLARRKPVLPALTINPTIAEGPSPTSEGASEANLVDLQKKLEELELDEQ
QKKRLEAFLTQKAKVGELKDDDFERISELGAGNGGVVTKVQHRPSGLIM
ARKLIHLEIKPAIRNQIIRELQVLHECNSPYIVGFYGAFYSDGEISICM
EHMDGGSLDQVLKEAKRIPEEILGKVSIAVLRGLAYLREKHQIMHRDVK
PSNILVNSRGEIKLCDFGVSGQLIDSMANSFVGTRSYMAPERLQGTHYS
VQSDIWSMGLSLVELAVGRYPIPPPDAKELEAIFGRPVVDGEEGEPHSI
SPRPRPPGRPVSGHGMDSRPAMAIFELLDYIVNEPPPKLPNGVFTPDFQ
EFVNKCLIKNPAERADLKMLTNHTFIKRSEVEEVDFAGWLCKTLRLNQP
GTPTRTAV

### ERK and MEK Inhibitors

ERK1/2 inhibitors and MEK inhibitors include any such inhibitor which inhibits ERK1 (also known as p44), ERK2 (also known as p42) or MEK (including MEK1 and MEK2) to any degree. In general, substances which inhibit ERK1 also inhibit ERK2, and vice versa. Such inhibitors may be referred to as ERK1/2 inhibitors or ERK inhibitors.

In an embodiment of the invention, the ERK1 or ERK2 inhibitor is any inhibitor set forth in published international patent application no. WO2007/070398.

In an embodiment of the invention an ERK1/2 inhibitor is any of the following:

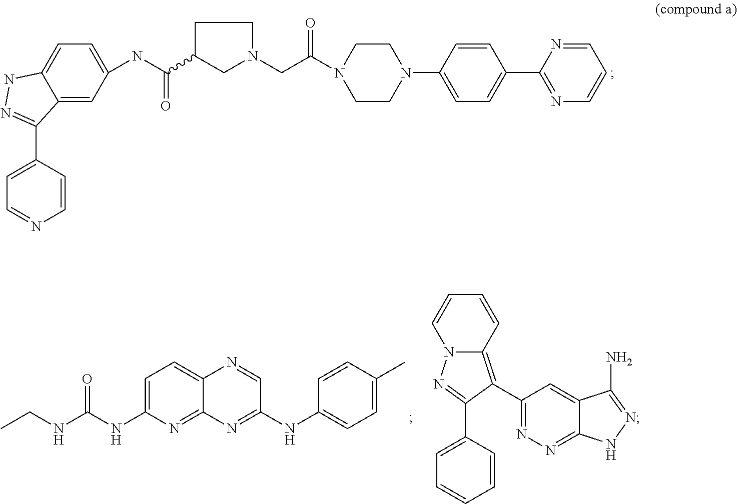

or
any other small molecule ERK1/2 inhibitor or antibody or antigen-binding fragment thereof which binds specifically to ERK1 or ERK2 and inhibits activity of such an enzyme to any degree whatsoever.

In an embodiment of the invention a MEK inhibitor is any of the following:

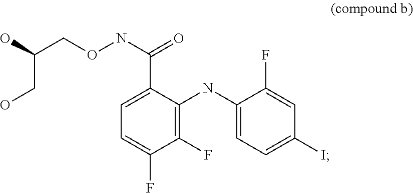

or

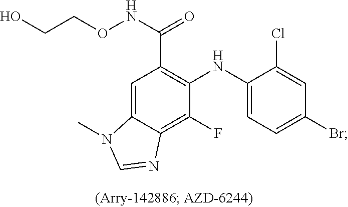

or any other small molecule MEK inhibitor any antibody or antigen-binding fragment thereof which binds specifically to MEK and inhibits activity of such an enzyme to any degree whatsoever.

In an embodiment of the invention, the ERK1 or ERK2 inhibitor is represented by the following structural formula (1.0):

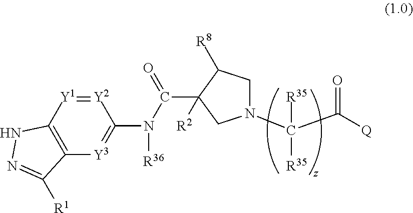

or any pharmaceutically acceptable salt thereof, wherein:

Y¹, Y², and Y³ are each independently selected from the group consisting of: —CH═, —N═ and —CR⁹═;

z is 1 to 3;

Q is a substituent selected from the group consisting of:

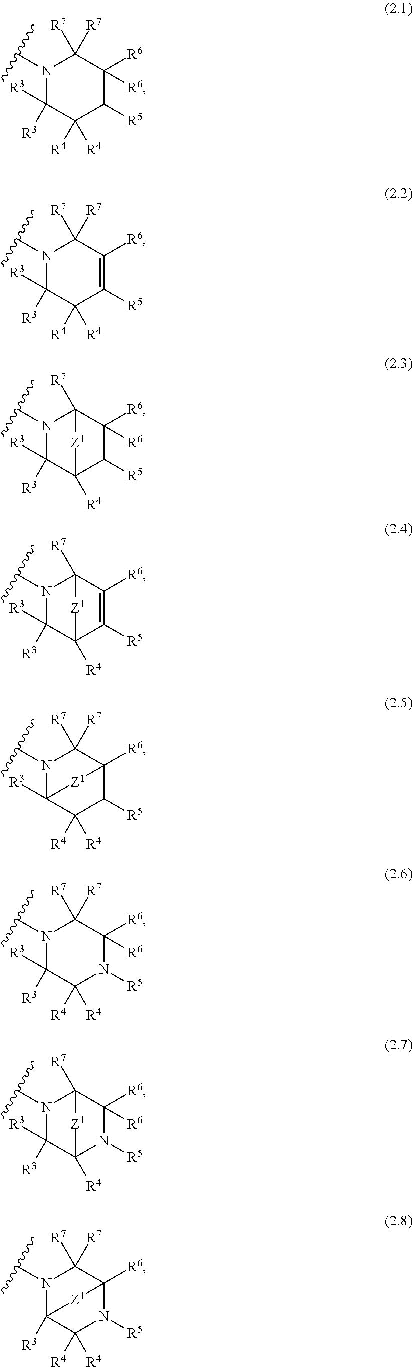

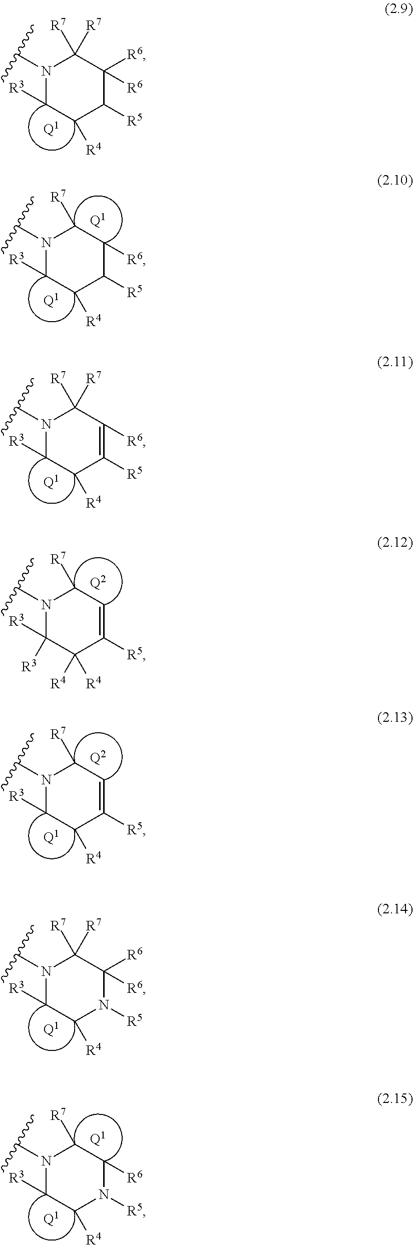

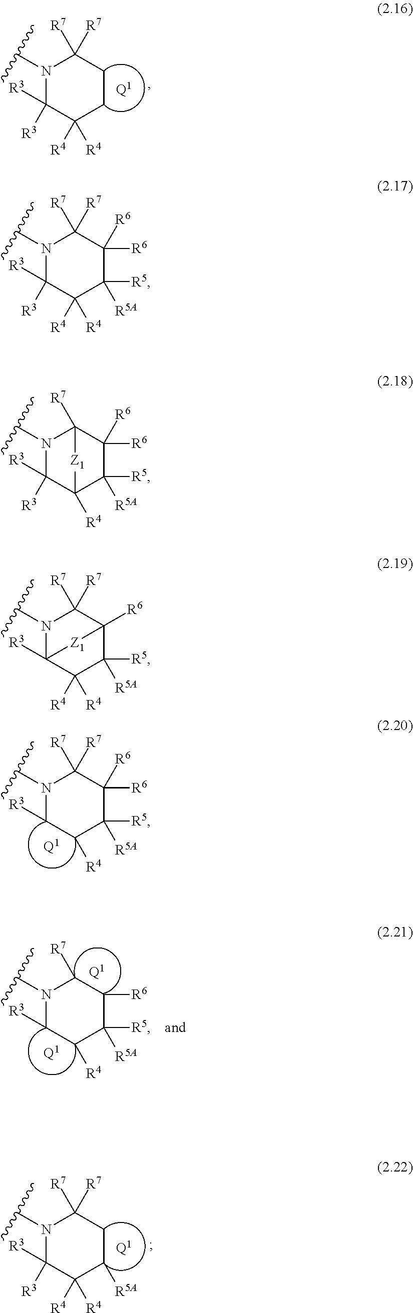

Each Q¹ represents a ring independently selected from the group consisting of: cycloalkyl, substituted cycloalkyl, heterocycloalkyl, substituted heterocycloalkyl, aryl, substituted aryl, heteroaryl, and substituted heteroaryl, wherein said substituted rings are substituted with 1 to 3 substituents independently selected from the group consisting of: the R¹⁰ moieties; provided that when Q¹ is aryl, heteroaryl, substituted aryl or substituted heteroaryl then the carbon atoms at the ring junction are not substituted;

Q² represents a ring selected from the group consisting of: cycloalkyl, substituted cycloalkyl, heterocycloalkyl, and substituted heterocycloalkyl, wherein said substituted rings are substituted with 1 to 3 substituents independently selected from the group consisting of: the R¹⁰ moieties;

Z¹ represents —(C(R²⁴)₂)_{w}— wherein each R²⁴ is independently selected from the group consisting of: H, alkyl and F, and wherein w is 1, 2 or 3;

Z² is selected from the group consisting of: —N(R⁴⁴)—, —O— and —C(R⁴⁶)₂—;

m is 1 to 6;

n is 1 to 6;

p is 0 to 6;

t is 0, 1, or 2;

R¹ is selected from the group consisting of:
(1) —CN,
(2) —NO₂,
(3) —OR¹⁰,
(4) —SR¹⁰,
(5) —N(R¹⁰)₂,
(6) R¹⁰,
(7) —C(O)R¹⁰,
(8) —(C(R³⁰)₂)ₙ—NR³²—C(O)—R¹⁰,
(9) —(C(R³)₂)ₙ—NR³²—S(O)ₜ—R¹⁰,
(10) —(C(R³⁰)₂)ₙ—NR³²—C(O)—N(R³²)—R¹⁰,
(11)

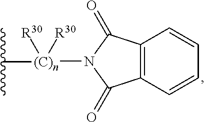

(12) —CF₃,
(13) —C(O)OR¹⁰,
(14) —(C(R³⁰)₂)ₙR¹³ (e.g., —(CH₂)ₙR¹³),
(15) alkenyl,
(16) —NR³²—C(O)—R¹⁴,
(17)

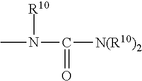

wherein each R¹⁰ is independently selected,
(18)

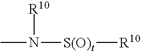

wherein each R¹⁰ is independently selected,
(19)

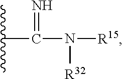

(20) —C(O)—NR³²—(C(R³⁰)₂)ₚ—OR¹⁰,
(21) —C(O)N(R¹⁰)₂ wherein each R¹⁰ is independently selected,
(22) —C(O)—NR³²—C(R¹⁸)₃,
(23) —C(O)—NR³²—(C(R³⁰)₂)ₙ—C(O)—N(R¹⁰)₂,
(24) heterocycloalkenyl,
(25)

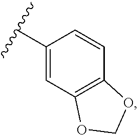

and
(26) arylalkenyl-;

R² is selected from the group consisting of:
(1) H,
(2) —CN,
(3) halo,
(4) alkyl,
(5) substituted alkyl wherein said substituted alkyl is substituted with 1 to 3 substituents selected from the group consisting of: (a) —OH, (b) —O-alkyl (e.g., —O—(C₁-C₃alkyl), (c) —O-alkyl substituted with 1 to 3 F atoms, and (d) —N(R⁴⁰)₂ wherein each R⁴⁰ is independently selected from the group consisting of: (i) H, (ii) C₁-C₃ alkyl, (iii) —CF₃, and (e) halo,
(6) alkynyl,
(7) alkenyl,
(8) —(CH₂)ₘR¹¹,
(9) —N(R²⁶)₂,
(10) —OR²³,
(11) —N(R²⁶)C(O)R⁴²,
(12) cycloalkyl,
(13) cycloalkylalkyl,
(14)

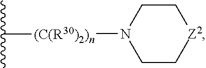

(15) —O-(substituted alkyl) wherein said substituted alkyl is substituted with 1 to 3 F atoms,
(16) —S(O)ₜ-alkyl,
(17) —C(O)-alkyl,
(18)

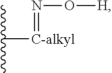

(19)

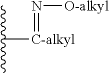

wherein each alkyl is independently selected,
(20)

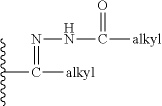

which each alkyl is independently selected,
(21)

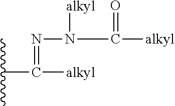

wherein each alkyl is independently selected,
(22) —N(R⁴⁸)—C(O)—R⁴⁸ wherein each R⁴⁸ is independently selected from the group consisting of: H and alkyl, and
(23) —C(O)-alkyl, such as, for example, —C(O)—(C₁-C₆ alkyl), such as, for example, —C(O)CH₃;

each R³, R⁴, R⁵, R⁶ and R⁷ is independently selected from the group consisting of:
(1) H,
(2) alkenyl,
(3) substituted alkenyl,
(4) alkyl,
(5) substituted alkyl,
(6) cycloalkyl,
(7) substituted cycloalkyl,
(8) cycloalkylalkyl-,
(9) substituted cycloalkylalkyl-,
(10) heterocycloalkyl,
(11) substituted heterocycloalkyl,
(12) heterocycloalkylalkyl-,
(13) substituted heterocycloalkylalkyl-,
(14) —C(O)R¹⁰,
(15) arylheteroaryl-,
(16) substituted arylheteroaryl-,
(17) heteroarylaryl-,
(18) substituted heteroarylaryl-,
(19) aryl,
(20) substituted aryl,
(21) heteroaryl,
(22) substituted heteroaryl,
(23) heteroarylheteroaryl-,
(24) substituted heteroarylheteroaryl-,
(25) arylaminoheteroaryl-,
(26) substituted arylaminoheteroaryl-,
(27) arylalkynyl-,
(28) substituted arylalkynyl-,
(29) heteroarylalkynyl-,
(30) substituted heteroarylalkynyl-,

wherein said R³, R⁴, R⁵, R⁶ and R⁷ substituted groups (7), (9), (11), (13), (16), (18), (20), (22), (24), (26), (28) and (30) are substituted with 1 to 3 substituents independently selected from the group consisting of: —NH₂, alkyl, alkenyl, halo, —C(O)—NH—R²⁸, —C(O)OR²⁸, and —C(O)R²⁸, and

wherein said R³, R⁴, R⁵, R⁶ and R⁷ substituted groups (3) and (5) are substituted with 1 to 3 substituents independently selected from the group consisting of: —NH₂, halo (e.g., F, Cl and Br, and in another example F), —C(O)—NH—R²⁸ (e.g., —C(O)—NH—CH₃), —C(O)OR²⁸ (e.g., —C(O)OC₂H₅), and —C(O)R²⁸ (e.g., —C(O)CH₃);

R^{5A} is selected from the group consisting of: halo, —OH, and —O-alkyl;

R⁸ is selected from the group consisting of: H, —OH, —N(R¹⁰)₂, —NR¹⁰C(O)R¹², and alkyl;

each R⁹ is independently selected from the group consisting of: halogen, —CN, —NO₂, —OR¹⁰, —SR¹⁰, —N(R¹⁰)₂, and R¹⁰;

each R¹⁰ is independently selected from the group consisting of: H, alkyl, aryl, arylalkyl, heteroaryl, heteroarylalkyl, cycloalkyl, cycloalkylalkyl, heterocycloalkyl, heterocycloalkylalkyl, alkylheteroaryl-, alkylaryl-, substituted alkyl, substituted aryl, substituted arylalkyl, substituted heteroaryl, substituted heteroarylalkyl, substituted cycloalkyl, substituted cycloalkylalkyl, substituted heterocycloalkyl, substituted heterocycloalkylalkyl, substituted alkylheteroaryl-, substituted alkylaryl-, heterocycloalkenyl, and substituted heterocycloalkenyl, and wherein:

said R¹⁰ substituted alkyl is substituted with 1 to 3 substituents independently selected from the group consisting of: —NH₂, —NHR²⁰, —NO₂, —CN, —OR²⁶, halo, —C(O)—NH—R²⁶, —C(O)OR²⁶, and —C(O)R²⁶, and

said R¹⁰ substituted aryl, substituted arylalkyl, substituted heteroaryl, substituted heteroarylalkyl, substituted cycloalkyl, substituted cycloalkylalkyl, substituted heterocycloalkyl, substituted heterocycloalkylalkyl, substituted alkylheteroaryl- and substituted alkylaryl- are substituted with 1 to 3 substituents independently selected from the group consisting of: (1) —NH₂, (2) —NO₂, (3) —CN,

(4) —OH, (5) —OR²⁰, (6) —OCF₃, (7) alkyl substituted with 1 to 3 independently selected halo atoms, (8) —C(O)R³⁸, (9) alkyl, (10) alkenyl, (11) halo, (12) —C(O)—NH—R²⁸,
(13) —C(O)OR³⁸, (14) —C(O)—NR³²—(C(R³⁰)₂)ₙ—N(R³⁸)₂, (15) —S(O)ₜR³⁸,

### (16) —C(O)—NR³²—R³⁸, (17) —NR³²—C(O)—R³⁸,

(18)

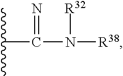

(19) —NHR²⁰, and (20) cycloalkyl;

R¹¹ is selected from the group consisting of: F, —OH, —CN, —OR¹⁰, —NHNR¹R¹⁰, —SR¹⁰ and heteroaryl;

R¹² is selected from the group consisting of: alkyl, aryl, heteroaryl, cycloalkyl, cycloalkylalkyl, heterocycloalkyl and heterocycloalkylalkyl;

R¹⁴ is selected from the group consisting of: alkyl, aryl, heteroaryl, cycloalkyl, cycloalkylalkyl-, heterocycloalkyl, alkylheterocycloalkyl, heterocycloalkylalkyl-, alkylheteroaryl- and alkylaryl-;

R¹⁵ is selected from the group consisting of: H, —OH, alkyl, aryl, heteroaryl, cycloalkyl, cycloalkylalkyl-, heterocycloalkyl and heterocycloalkylalkyl-, alkylheteroaryl- and alkylaryl-;

R²⁰ represents alkyl;

R²³ is selected from the group consisting of: H, alkyl, aryl, cycloalkyl, and cycloalkylalkyl-;

each R²⁶ is independently selected from the group consisting of: H and alkyl;

R²⁸ is alkyl;

each R³⁰ is independently selected from the group consisting of: H, alkyl, and F;

each R³² is independently selected from the group consisting of: H and alkyl, and wherein each R³² is generally H;

each R³⁵ is independently selected from the group consisting of: H and C₁ to C₆ alkyl;

R³⁶ is selected from the group consisting of: H, alkyl, and —O-alkyl;

each R³⁸ is independently selected from the group consisting of: H, alkyl, aryl, arylalkyl, heteroaryl, heteroarylalkyl, cycloalkyl, cycloalkylalkyl, heterocycloalkyl, heterocycloalkylalkyl, alkylheteroaryl-, alkylaryl-, substituted alkyl, substituted aryl, substituted arylalkyl, substituted heteroaryl, substituted heteroarylalkyl, substituted cycloalkyl, substituted cycloalkylalkyl, substituted heterocycloalkyl, substituted heterocycloalkylalkyl, substituted alkylheteroaryl- and substituted alkylaryl-, and wherein:

said R³⁸ substituted alkyl is substituted with 1 to 3 substituents independently selected from the group consisting of: —NH₂, —NO₂, —CN, —OR²⁶, halo, —C(O)—NH—R²⁸, —C(O)OR²⁸, and —C(O)R²⁸, and

said R³⁸ substituted aryl, substituted arylalkyl, substituted heteroaryl, substituted heteroarylalkyl, substituted cycloalkyl, substituted cycloalkylalkyl, substituted heterocycloalkyl, substituted heterocycloalkylalkyl, substituted alkylheteroaryl- and substituted alkylaryl- are substituted with 1 to 3 substituents independently selected from the group consisting of: (1) —NH₂, (2) —NO₂, (3) —CN,

(4) —OH, (5) —OR²⁰, (6) —OCF₃, (7) —CF₃, (8) —C(O)R²⁶, (9) alkyl, (10) alkenyl, (11) halo, (12) —C(O)—NH—R²⁶, (13) —C(O)OR²⁶, (14) —C(O)—NR³²—(C(R³⁰)₂)ₙ—N(R²⁶)₂,

### (15) —S(O)ₜR²⁶, (16) —C(O)N(R³²)(R²⁶), (17) —NR³²C(O)R²⁶,

(18)

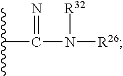

and

### (19) —NHR²⁰;

R⁴² is selected from the group consisting of: alkyl, aryl, heteroaryl, and cycloalkyl;

R⁴⁴ is selected from the group consisting of: H, alkyl, cycloalkyl, and cycloalkylalkyl; and

Each R⁴⁶ is independently selected from the group consisting of: H, alkyl, cycloalkyl, and cycloalkylalkyl.

As used herein, unless otherwise specified, the following terms have the following meanings, and unless otherwise specified, the definitions of each term (i.e., moiety or substituent) apply when that term is used individually or as a component of another term (e.g., the definition of aryl is the same for aryl and for the aryl portion of arylalkyl, alkylaryl, arylalkynyl, and the like):
“acyl” means an H—C(O)—, alkyl-C(O)—, alkenyl-C(O)—, Alkynyl-C(O)—, cycloalkyl-C(O)—, cycloalkenyl-C(O)—, or cycloalkynyl-C(O)— group in which the various groups are as defined below (and as defined below, the alkyl, alkenyl, alkynyl, cycloalkyl, cycloalkenyl and cycloalkynyl moieties can be substituted); the bond to the parent moiety is through the carbonyl; preferred acyls contain a lower alkyl; Non-limiting examples of suitable acyl groups include formyl, acetyl, propanoyl, 2-methylpropanoyl, butanoyl and cyclohexanoyl;
“alkenyl” means an aliphatic hydrocarbon group (chain) comprising at least one carbon to carbon double bond, wherein the chain can be straight or branched, and wherein said group comprises about 2 to about 15 carbon atoms; Preferred alkenyl groups comprise about 2 to about 12 carbon atoms in the chain; and more preferably about 2 to about 6 carbon atoms in the chain; branched means that one or more lower alkyl groups, such as methyl, ethyl or propyl, or alkenyl groups are attached to a linear alkenyl chain; “lower alkenyl” means an alkenyl group comprising about 2 to about 6 carbon atoms in the chain, and the chain can be straight or branched; the term “substituted alkenyl” means that the alkenyl group is substituted by one or more independently selected substituents, and each substituent is independently selected from the group consisting of: halo, alkyl, aryl, cycloalkyl, cyano, alkoxy and —S(alkyl); non-limiting examples of suitable alkenyl groups include ethenyl, propenyl, n-butenyl, 3-methylbut-2-enyl, n-pentenyl, octenyl and decenyl;
“alkoxy” means an alkyl-O— group (i.e., the bond to the parent moiety is through the ether oxygen) in which the alkyl group is unsubstituted or substituted as described below; non-limiting examples of suitable alkoxy groups include methoxy, ethoxy, n-propoxy, isopropoxy, n-butoxy and heptoxy;
“alkoxycarbonyl” means an alkyl-O—CO— group (i.e., the bond to the parent moiety is through the carbonyl) wherein the alkyl group is unsubstituted or substituted as previously defined; non-limiting examples of suitable alkoxycarbonyl groups include methoxycarbonyl and ethoxycarbonyl;
“alkyl” (including the alkyl portions of other moieties, such as trifluoroalkyl and alkyloxy) means an aliphatic hydrocarbon group (chain) that can be straight or branched wherein said group comprises about 1 to about 20 carbon atoms in the chain; preferred alkyl groups comprise about 1 to about 12 carbon atoms in the chain; more preferred alkyl groups comprise about 1 to about 6 carbon atoms in the chain; branched means that one or more lower alkyl groups, such as methyl, ethyl or propyl, are attached to a linear alkyl chain; “lower alkyl” means a group comprising about 1 to about 6 carbon atoms in the chain, and said chain can be straight or branched; the term “substituted alkyl” means that the alkyl group is substituted by one or more independently selected substituents, and wherein each substituent is independently selected from the group consisting of: halo, aryl, cycloalkyl, cyano, hydroxy, alkoxy, alkylthio, amino, —NH(alkyl), —NH(cycloalkyl), —N(alkyl)₂, carboxy, —C(O)O-alkyl and
   —S(alkyl); non-limiting examples of suitable alkyl groups include methyl, ethyl, n-propyl, isopropyl, n-butyl, t-butyl, n-pentyl, heptyl, nonyl, decyl, fluoromethyl, trifluoromethyl and cyclopropylmethyl;
“alkylaryl” (or alkaryl) means an alkyl-aryl- group (i.e., the bond to the parent moiety is through the aryl group) wherein the alkyl group is unsubstituted or substituted as defined above, and the aryl group is unsubstituted or substituted as defined below; preferred alkylaryls comprise a lower alkyl group; non-limiting examples of suitable alkylaryl groups include o-tolyl, p-tolyl and xylyl;
“alkylheteroaryl” means an alkyl-heteroaryl- group (i.e., the bond to the parent moiety is through the heteroaryl group) wherein the alkyl is unsubstituted or substituted as defined above and the heteroaryl group is unsubstituted or substituted as defined below;
“alkylsulfinyl” means an alkyl-S(O)— group (i.e., the bond to the parent moiety is through the sulfinyl) wherein the alkyl group is unsubstituted or substituted as previously defined; preferred groups are those in which the alkyl group is lower alkyl;
“alkylsulfonyl” means an alkyl-S(O₂)— group (i.e., the bond to the parent moiety is through the sulfonyl) wherein the alkyl group is unsubstituted or substituted as previously defined; preferred groups are those in which the alkyl group is lower alkyl;
“alkylthio” means an alkyl-S— group (i.e., the bond to the parent moiety is through the sulfur) wherein the alkyl group is unsubstituted or substituted as previously described; non-limiting examples of suitable alkylthio groups include methylthio, ethylthio, i-propylthio and heptylthio;
“alkynyl” means an aliphatic hydrocarbon group (chain) comprising at least one carbon to carbon triple bond, wherein the chain can be straight or branched, and wherein the group comprises about 2 to about 15 carbon atoms in the; preferred alkynyl groups comprise about 2 to about 12 carbon atoms in the chain; and more preferably about 2 to about 4 carbon atoms in the chain; Branched means that one or more lower alkyl groups, such as methyl, ethyl or propyl, are attached to a linear alkynyl chain; “lower alkynyl” means an alkynyl group comprising about 2 to about 6 carbon atoms in the chain, and the chain can be straight or branched; non-limiting examples of suitable alkynyl groups include ethynyl, propynyl, 2-butynyl, 3-methylbutynyl, n-pentynyl, and decynyl; the term “substituted alkynyl” means that the alkynyl group is substituted by one or more independently selected, and each substituent is independently selected from the group consisting of alkyl; aryl and cycloalkyl;
“amino means a —NH₂ group;
“aralkenyl” (or arylalkenyl) means an aryl-alkenyl- group (i.e., the bond to the parent moiety is through the alkenyl group) wherein the aryl group is unsubstituted or substituted as defined below, and the alkenyl group is unsubstituted or substituted as defined above; preferred aralkenyls contain a lower alkenyl group; non-limiting examples of suitable aralkenyl groups include 2-phenethenyl and 2-naphthylethenyl;
“aralkyl” (or arylalkyl) means an aryl-alkyl- group (i.e., the bond to the parent moiety is through the alkyl group) wherein the aryl is unsubstituted or substituted as defined below and the alkyl is unsubstituted or substituted as defined above; preferred aralkyls comprise a lower alkyl group; non-limiting examples of suitable aralkyl groups include benzyl, 2-phenethyl and naphthalenylmethyl;
“aralkyloxy” (or arylalkyloxy) means an aralkyl-O— group (i.e., the bond to the parent moiety is through the ether oxygen) wherein the aralkyl group is unsubstituted or substituted as previously described; non-limiting examples of suitable aralkyloxy groups include benzyloxy and 1- or 2-naphthalenemethoxy;
“aralkoxycarbonyl” means an aralkyl-O—C(O)— group (i.e., the bond to the parent moiety is through the carbonyl) wherein the aralkyl group is unsubstituted or substituted as previously defined; a non-limiting example of a suitable aralkoxycarbonyl group is benzyloxycarbonyl;
“aralkylthio” means an aralkyl-S— group (i.e., the bond to the parent moiety is through the sulfur) wherein the aralkyl group is unsubstituted or substituted as previously described; a non-limiting example of a suitable aralkylthio group is benzylthio;
“aroyl” means an aryl-C(O)— group (i.e., the bond to the parent moiety is through the carbonyl) wherein the aryl group is unsubstituted or substituted as defined below; non-limiting examples of suitable groups include benzoyl and 1- and 2-naphthoyl;
“aryl” (sometimes abbreviated “ar”) means an aromatic monocyclic or multicyclic ring system comprising about 6 to about 14 carbon atoms, preferably about 6 to about 10 carbon atoms; the aryl group can be optionally substituted with one or more independently selected “ring system substituents” (defined below). Non-limiting examples of suitable aryl groups include phenyl and naphthyl;
“arylalkynyl” means an aryl-alkynyl- group (i.e., the bond to the parent moiety is through the alkynyl group) wherein the aryl group is unsubstituted or substituted as defined above, and the alkynyl group is unsubstituted or substituted as defined above;
“arylaminoheteroaryl” means an aryl-amino-heteroaryl group (i.e., the bond to the parent moiety is through the heteroaryl group) wherein the aryl group is unsubstituted or substituted as defined above, the amino group is as defined above (i.e., a —NH— here), and the heteroaryl group is unsubstituted or substituted as defined below;
“arylheteroaryl” means an aryl-heteroaryl group—(i.e., the bond to the parent moiety is through the heteroaryl group) wherein the aryl group is unsubstituted or substituted as defined above, and the heteroaryl group is unsubstituted or substituted as defined below;
“aryloxy” means an aryl-O— group (i.e., the bond to the parent moiety is through the ether oxygen) wherein the aryl group is unsubstituted or substituted as defined above; non-limiting examples of suitable aryloxy groups include phenoxy and naphthoxy;
“aryloxycarbonyl” means an aryl-O—C(O)— group (i.e., the bond to the parent moiety is through the carbonyl) wherein the aryl group is unsubstituted or substituted as previously defined; non-limiting examples of suitable aryloxycarbonyl groups include phenoxycarbonyl and naphthoxycarbonyl;
“arylsulfinyl” means an aryl-S(O)— group (i.e., the bond to the parent moiety is through the sulfinyl) wherein aryl is unsubstituted or substituted as previously defined;
“arylsulfonyl” means an aryl-S(O₂)— group (i.e., the bond to the parent moiety is through the sulfonyl) wherein aryl is unsubstituted or substituted as previously defined;
“arylthio” means an aryl-S— group (i.e., the bond to the parent moiety is through the sulfur) wherein the aryl group is unsubstituted or substituted as previously described; non-limiting examples of suitable arylthio groups include phenylthio and naphthylthio;
“cycloalkenyl” means a non-aromatic mono or multicyclic ring system comprising about 3 to about 10 carbon atoms, preferably about 5 to about 10 carbon atoms that contains at least one carbon-carbon double bond; preferred cycloalkenyl rings contain about 5 to about 7 ring atoms; the cycloalkenyl can be optionally substituted with one or more independently selected “ring system substituents” (defined below); Non-limiting examples of suitable monocyclic cycloalkenyls include cyclopentenyl, cyclohexenyl, cycloheptenyl, and the like; a non-limiting example of a suitable multicyclic cycloalkenyl is norbornylenyl;
“cycloalkyl” means a non-aromatic mono- or multicyclic ring system comprising about 3 to about 7 carbon atoms, preferably about 3 to about 6 carbon atoms; the cycloalkyl can be optionally substituted with one or more independently selected “ring system substituents” (defined below); non-limiting examples of suitable monocyclic cycloalkyls include cyclopropyl, cyclopentyl, cyclohexyl, cycloheptyl and the like; non-limiting examples of suitable multicyclic cycloalkyls include 1-decalin, norbornyl, adamantyl and the like;
“cycloalkylalkyl” means a cycloalkyl-alkyl- group (i.e., the bond to the parent moiety is through the alkyl group) wherein the cycloalkyl moiety is unsubstituted or substituted as defined above, and the alkyl moiety is unsubstituted or substituted as defined above;
“halo” means fluoro, chloro, bromo, or iodo groups; preferred halos are fluoro, chloro or bromo, and more preferred are fluoro and chloro;
“halogen” means fluorine, chlorine, bromine, or iodine; preferred halogens are fluorine, chlorine and bromine;
“haloalkyl” means an alkyl, as defined above, wherein one or more hydrogen atoms on the alkyl is replaced by a halo group, as defined above;
“heteroaralkenyl” means a heteroaryl-alkenyl- group (i.e., the bond to the parent moiety is through the alkenyl group) wherein the heteroaryl group is unsubstituted or substituted as defined below, and the alkenyl group is unsubstituted or substituted as defined above;
“heteroaralkyl” (or heteroarylalkyl) means a heteroaryl-alkyl- group (i.e., the bond to the parent moiety is through the alkyl group) in which the heteroaryl is unsubstituted or substituted as defined below, and the alkyl group is unsubstituted or substituted as defined above; preferred heteroaralkyls comprise an alkyl group that is a lower alkyl group; non-limiting examples of suitable aralkyl groups include pyridylmethyl, 2-(furan-3-yl)ethyl and quinolin-3-ylmethyl;
“heteroaralkylthio” means a heteroaralkyl-S— group wherein the heteroaralkyl group is unsubstituted or substituted as defined above;
“heteroaryl” means an aromatic monocyclic or multicyclic ring system comprising about 5 to about 14 ring atoms, preferably about 5 to about 10 ring atoms, in which one or more of the ring atoms is an element other than carbon, for example nitrogen, oxygen or sulfur, alone or in combination; preferred heteroaryls comprise about 5 to about 6 ring atoms; the “heteroaryl” can be optionally substituted by one or more independently selected “ring system substituents” (defined below); the prefix aza, oxa or thia before the heteroaryl root name means that at least a nitrogen, oxygen or sulfur atom, respectively, is present as a ring atom; a nitrogen atom of a heteroaryl can be optionally oxidized to the corresponding N-oxide; non-limiting examples of suitable heteroaryls include pyridyl, pyrazinyl, furanyl, thienyl, pyrimidinyl, isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like;
“heteroarylalkynyl” (or heteroaralkynyl) means a heteroaryl-alkynyl- group (i.e., the bond to the parent moiety is through the alkynyl group) wherein the heteroaryl group is unsubstituted or substituted as defined above, and the alkynyl group is unsubstituted or substituted as defined above;
“heteroarylaryl” (or heteroararyl) means a heteroaryl-aryl- group (i.e., the bond to the parent moiety is through the aryl group) wherein the heteroaryl group is unsubstituted or substituted as defined above, and the aryl group is unsubstituted or substituted as defined above;
“heteroarylheteroarylaryl” means a heteroaryl-heteroaryl- group (i.e., the bond to the parent moiety is through the last heteroaryl group) wherein each heteroaryl group is independently unsubstituted or substituted as defined above;
“heteroarylsulfinyl” means a heteroaryl-SO— group wherein the heteroaryl group is unsubstituted or substituted as defined above;
“heteroarylsulfonyl” means a heteroaryl-SO₂— group wherein the heteroaryl group is unsubstituted or substituted as defined above;
“heteroarylthio” means a heteroaryl-S— group wherein the heteroaryl group is unsubstituted or substituted as defined above;
“heterocyclenyl” (or heterocycloalkenyl) means a non-aromatic monocyclic or multicyclic ring system comprising about 3 to about 10 ring atoms, preferably about 5 to about 10 ring atoms, in which one or more of the atoms in the ring system is an element other than carbon (for example one or more heteroatoms independently selected from the group consisting of nitrogen, oxygen and sulfur atom), and which contains at least one carbon-carbon double bond or carbon-nitrogen double bond; there are no adjacent oxygen and/or sulfur atoms present in the ring system; Preferred heterocyclenyl rings contain about 5 to about 6 ring atoms; the prefix aza, oxa or thia before the heterocyclenyl root name means that at least a nitrogen, oxygen or sulfur atom, respectively, is present as a ring atom; the heterocyclenyl can be optionally substituted by one or more independently selected “Ring system substituents” (defined below); the nitrogen or sulfur atom of the heterocyclenyl can be optionally oxidized to the corresponding N-oxide, S-oxide or S,S-dioxide; non-limiting examples of suitable monocyclic azaheterocyclenyl groups include 1,2,3,4-tetrahydropyridine, 1,2-dihydropyridyl, 1,4-dihydropyridyl, 1,2,3,6-tetrahydropyridine, 1,4,5,6-tetrahydropyrimidine, 2-pyrrolinyl, 3-pyrrolinyl, 2-imidazolinyl, 2-pyrazolinyl, and the like; Non-limiting examples of suitable oxaheterocyclenyl groups include 3,4-dihydro-2H-pyran, dihydrofuranyl, fluorodihydrofuranyl, and the like; A non-limiting example of a suitable multicyclic oxaheterocyclenyl group is 7-oxabicyclo[2.2.1]heptenyl; non-limiting examples of suitable monocyclic thiaheterocyclenyl rings include dihydrothiophenyl, dihydrothiopyranyl, and the like;
“heterocycloalkylalkyl” (or heterocyclylalkyl) means a heterocycloalkyl-alkyl- group (i.e., the bond to the parent moiety is through the alkyl group) wherein the heterocycloalkyl group (i.e., the heterocyclyl group) is unsubstituted or substituted as defined below, and the alkyl group is unsubstituted or substituted as defined above;
“heterocyclyl” (or heterocycloalkyl) means a non-aromatic saturated monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur, alone or in combination; there are no adjacent oxygen and/or sulfur atoms present in the ring system; preferred heterocyclyls contain about 5 to about 6 ring atoms; the prefix aza, oxa or thia before the heterocyclyl root name means that at least a nitrogen, oxygen or sulfur atom respectively is present as a ring atom; the heterocyclyl can be optionally substituted by one or more independently selected “ring system substituents” (defined below); the nitrogen or sulfur atom of the heterocyclyl can be optionally oxidized to the corresponding N-oxide, S-oxide or S,S-dioxide; non-limiting examples of suitable monocyclic heterocyclyl rings include piperidyl, pyrrolidinyl, piperazinyl, morpholinyl, thiomorpholinyl, thiazolidinyl, 1,3-dioxolanyl, 1,4-dioxanyl, tetrahydrofuranyl, tetrahydrothiophenyl, tetrahydrothiopyranyl, and the like;
“hydroxyalkyl” means a HO-alkyl- group wherein the alkyl group is substituted or unsubstituted as defined above; preferred hydroxyalkyls comprise a lower alkyl; Non-limiting examples of suitable hydroxyalkyl groups include hydroxymethyl and 2-hydroxyethyl; and
“ring system substituent” means a substituent attached to an aromatic or non-aromatic ring system that, for example, replaces an available hydrogen on the ring system; ring system substituents are each independently selected from the group consisting of: alkyl, aryl, heteroaryl, aralkyl, alkylaryl, aralkenyl, heteroaralkyl, alkylheteroaryl, heteroaralkenyl, hydroxy, hydroxyalkyl, alkoxy, aryloxy, aralkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylsulfinyl, arylsulfinyl, heteroarylsulfinyl, alkylthio, arylthio, heteroarylthio, aralkylthio, heteroaralkylthio, cycloalkyl, cycloalkenyl, heterocyclyl, heterocyclenyl, R⁶⁰R⁶⁵N—, R⁶⁰R⁶⁵N-alkyl-, R⁶⁰R⁶⁵NC(O)— and R⁶OR⁶⁵NSO₂—, wherein R⁶⁰ and R⁶⁵ are each independently selected from the group consisting of: hydrogen, alkyl, aryl, and aralkyl; “Ring system substituent” also means a cyclic ring of 3 to 7 ring atoms, wherein 1-2 ring atoms can be heteroatoms, attached to an aryl, heteroaryl, heterocyclyl or heterocyclenyl ring by simultaneously substituting two ring hydrogen atoms on said aryl, heteroaryl, heterocyclyl or heterocyclenyl ring; Non-limiting examples include:

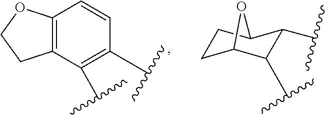

and the like.

Lines drawn into a ring mean that the indicated bond may be attached to any of the substitutable ring carbon atoms.

Any carbon or heteroatom with unsatisfied valences in the text, schemes, examples, structural formulae, and any Tables herein is assumed to have the hydrogen atom or atoms to satisfy the valences.

One or more compounds of the invention may also exist as, or optionally be converted to, a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTech., 5(1), article 12 (2004); and A. L. Bingham et al, Chem. Commun., 603-604 (2001). A typical, non-limiting, process involves dissolving the inhibitor compound in desired amounts of the desired solvent (organic or water or mixtures thereof) at a higher than ambient temperature, and cooling the solution at a rate sufficient to form crystals which are then isolated by standard methods. Analytical techniques such as, for example I.R. spectroscopy, show the presence of the solvent (or water) in the crystals as a solvate (or hydrate).

### Therapeutic Methods and Administration

The ERK1 and ERK2 and MEK inhibitors set forth herein may be used to treat any hyperproliferative disorder such as cancer. Cancers treatable using an inhibitor set forth herein include, e.g., choloangiocarcinoma, lung cancer, pancreatic cancer, colon cancer, myeloid leukemias, thyroid cancer, myelodysplastic syndrome, bladder carcinoma, epidermal carcinoma, melanoma, breast cancer, prostate cancer, head and neck cancers, ovarian cancer, brain cancers, cancers of mesenchymal origin, sarcomas, tetracarcinomas, neuroblastomas, kidney carcinomas, hepatomas, non-Hodgkin's lymphoma, multiple myeloma, and anaplastic thyroid carcinoma.

Melanoma treatable by administration of an ERK1 or ERK2 or MEK inhibitor discussed herein includes any stage or type of the disease. The inhibitors may be used to treat melanoma on any part of the body of a subject. For example, the inhibitors discussed herein may be used to treat lentigo maligna type melanoma, superficial spreading type melanoma, nodular type melanoma and acral-lentiginous type melanoma.

The amount and frequency of administration of the ERK1 and ERK2 or MEK inhibitors discussed herein and/or the pharmaceutically acceptable salts thereof will be regulated according to the judgment of the attending clinician considering such factors as age, condition and size of the patient as well as severity of the symptoms being treated. A typical therapeutically effective daily dosage regimen for oral administration can range from about 0.04 mg/day to about 4000 mg/day.

Typically, the administration and dosage of any agent (e.g., a further chemotherapeutic agent as discussed herein) is, when possible, done according to the schedule listed in the product information sheet of the approved agents, in the Physicians' Desk Reference 2003 (Physicians' Desk Reference, 57th Ed); Medical Economics Company; ISBN: 1563634457; 57th edition (November 2002), as well as therapeutic protocols known in the art.

The actual dosage employed may be varied depending upon the requirements of the patient and the severity of the condition being treated. Determination of the proper dosage regimen for a particular situation is within the skill in the art. For convenience, the total daily dosage may be divided and administered in portions during the day as required.

For preparing pharmaceutical compositions from the compounds described herein, inert, pharmaceutically acceptable carriers can be solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g., magnesium carbonate, magnesium stearate, talc, sugar or lactose. Tablets, powders, cachets and capsules can be used as solid dosage forms suitable for oral administration. For general information concerning formulations, see, e.g., Gilman, et al., (eds.) (1990), The Pharmacological Bases of Therapeutics, 8th Ed., Pergamon Press; A. Gennaro (ed.), Remington's Pharmaceutical Sciences, 18th Edition, (1990), Mack Publishing Co., Easton, Pa.; Avis, et al., (eds.) (1993) Pharmaceutical Dosage Forms: Parenteral Medications Dekker, New York; Lieberman, et al., (eds.) (1990) Pharmaceutical Dosage Forms: Tablets Dekker, New York; and Lieberman, et al., (eds.) (1990), Pharmaceutical Dosage Forms: Disperse Systems Dekker, New York, Kenneth A. Walters (ed.) (2002) Dermatological and Transdermal Formulations (Drugs and the Pharmaceutical Sciences), Vol 119, Marcel Dekker.

Liquid form preparations include solutions, suspensions and emulsions. As an example, water or water-propylene glycol solutions for parenteral injection or addition of sweeteners and opacifiers for oral solutions, suspensions and emulsions form part of the present invention. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g., nitrogen.

Also included are solid form preparations which are intended to be converted, shortly before use, to liquid form preparations for either oral or parenteral administration. Such liquid forms include solutions, suspensions and emulsions.

The ERK1 and ERK2 and MEK inhibitors discussed herein may also be deliverable transdermally. The transdermal compositions can take the form of creams, lotions, aerosols and/or emulsions and can be included in a transdermal patch of the matrix or reservoir type as are conventional in the art for this purpose.

In an embodiment of the invention, the inhibitor is administered orally.

In an embodiment of the invention, the pharmaceutical preparation is in a unit dosage form. In such form, the preparations subdivided into suitably sized unit doses containing appropriate quantities of the active component, e.g., an effective amount to achieve the desired purpose.

The quantity of active compound in a unit dose of preparation may be varied or adjusted from about 0.01 mg to about 1000 mg, preferably from about 0.01 mg to about 750 mg, more preferably from about 0.01 mg to about 500 mg, and most preferably from about 0.01 mg to about 250 mg according to the particular application.

### Further Chemotherapeutic Agents

Embodiments of the invention include methods of treating a medical condition such as cancer by administering an ERK1 or ERK2 or MEK inhibitor in association with any further chemotherapeutic agent (e.g., an anti-cancer therapeutic agent) or with a therapeutic procedure (e.g., anti-cancer radiation therapy or surgical tumorectomy).

Further chemotherapeutic agents include, for example, microtubule affecting agents, alkylating agents, antimetabolites, natural products and their derivatives, hormones and steroids (including synthetic analogs), and synthetics.

Examples of alkylating agents (including nitrogen mustards, ethylenimine derivatives, alkyl sulfonates, nitrosoureas and triazenes) include: Uracil mustard, Chlormethine, Cyclophosphamide (Cytoxan®), Ifosfamide, Melphalan, Chlorambucil, Pipobroman, Triethylene-melamine, Triethylenethiophosphoramine, Busulfan, Carmustine, Lomustine, Streptozocin, Dacarbazine, and Temozolomide.

Examples of antimetabolites (including folic acid antagonists, pyrimidine analogs, purine analogs and adenosine deaminase inhibitors) include: Methotrexate, 5-Fluorouracil, Floxuridine, Cytarabine, 6-Mercaptopurine, 6-Thioguanine, Fludarabine phosphate, Pentostatine, and Gemcitabine.

Examples of natural products and their derivatives (including vinca alkaloids, antitumor antibiotics, enzymes, lymphokines and epipodophyllotoxins) include: Vinblastine, Vincristine, Vindesine, Bleomycin, Dactinomycin, Daunorubicin, Doxorubicin, Epirubicin, Idarubicin, Paclitaxel (paclitaxel is a microtubule affecting agent and is commercially available as Taxol®), Paclitaxel derivatives (e.g. taxotere), Mithramycin, Deoxyco-formycin, Mitomycin-C, L-Asparaginase, Interferons (especially IFN-a), Etoposide, and Teniposide.

Examples of hormones and steroids (including synthetic analogs) include: 17α-Ethinylestradiol, Diethylstilbestrol, Testosterone, Prednisone, Fluoxymesterone, Dromostanolone propionate, Testolactone, Megestrolacetate, Tamoxifen, Methylprednisolone, Methyl-testosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, and Zoladex.

Examples of synthetics (including inorganic complexes such as platinum coordination complexes): Cisplatin, Carboplatin, Hydroxyurea, Amsacrine, Procarbazine, Mitotane, Mitoxantrone, Levamisole, and Hexamethylmelamine.

Examples of other chemotherapeutics include: Navelbene, CPT-11, Anastrazole, Letrazole, Capecitabinbe, Reloxafine, and Droloxafine.

A microtubule affecting agent (e.g., paclitaxel, a paclitaxel derivative or a paclitaxel-like compound), as used herein, includes a compound that affects microtubule formation and/or depolymerization and/or action. Such agents can be, for instance, microtubule stabilizing agents or agents which disrupt microtubule formation.

Microtubule affecting agents, useful in the methods of this invention, are well known to those skilled in the art and include, but are not limited to: Allocolchicine (NSC 406042), Halichondrin B (NSC 609395), Colchicine (NSC 757), Colchicine derivatives (e.g., NSC 33410), Dolastatin 10 (NSC 376128), Maytansine (NSC 153858), Rhizoxin (NSC 332598), Paclitaxel (Taxol®, NSC 125973), Paclitaxel derivatives (e.g., Taxotere, NSC 608832), Thiocolchicine (NSC 361792), Trityl Cysteine (NSC 83265), Vinblastine Sulfate (NSC 49842), Vincristine Sulfate (NSC 67574), Epothilone A, Epothilone, Discodermolide (see Service, (1996) Science, 274:2009), Estramustine, Nocodazole, MAP4, and the like. Examples of such agents are described in, for example, Bulinski (1997) J. Cell Sci. 110:3055-3064, Panda (1997) Proc. Natl. Acad. Sci. USA 94:10560-10564, Muhlradt (1997) Cancer Res. 57:3344-3346, Nicolaou (1997) Nature 387:268-272, Vasquez (1997) Mol. Biol. Cell. 8:973-985, and Panda (1996) J. Biol. Chem. 271:29807-29812.

Chemotherapeutic agents with paclitaxel-like activity include, but are not limited to, paclitaxel and paclitaxel derivatives (paclitaxel-like compounds) and analogues. Paclitaxel and its derivatives (e.g., Taxol and Taxotere) are available commercially. In addition, methods of making paclitaxel and paclitaxel derivatives and analogues are well known to those of skill in the art (see, e.g., U.S. Pat. Nos. 5,569,729; 5,565,478; 5,530,020; 5,527,924; 5,508,447; 5,489,589; 5,488,116; 5,484,809; 5,478,854; 5,478,736; 5,475,120; 5,468,769; 5,461,169; 5,440,057; 5,422,364; 5,411,984; 5,405,972; and 5,296,506).

More specifically, the term “paclitaxel” as used herein refers to the drug commercially available as Taxol® (NSC number: 125973). Taxol® inhibits eukaryotic cell replication by enhancing polymerization of tubulin moieties into stabilized microtubule bundles that are unable to reorganize into the proper structures for mitosis. Of the many available chemotherapeutic drugs, paclitaxel has generated interest because of its efficacy in clinical trials against drug-refractory tumors, including ovarian and mammary gland tumors (Hawkins (1992) Oncology, 6: 17-23, Horwitz (1992) Trends Pharmacol. Sci. 13: 134-146, Rowinsky (1990) J. Natl. Canc. Inst. 82: 1247-1259).

Additional microtubule affecting agents can be assessed using one of many such assays known in the art, e.g., a semiautomated assay which measures the tubulin-polymerizing activity of paclitaxel analogs in combination with a cellular assay to measure the potential of these compounds to block cells in mitosis (see Lopes (1997) Cancer Chemother. Pharmacol. 41:37-47).

Other further chemotherapeutic agents include cetuximab, erlotinib and gefitinib.

The term “in association with” indicates that the components of a composition of the invention can be formulated into a single composition for simultaneous delivery or formulated separately into two or more compositions (e.g., a kit). Furthermore, each component can be administered to a subject at a different time than when the other component is administered; for example, each administration may be given non-simultaneously (e.g., separately or sequentially) at several intervals over a given period of time. Moreover, the separate components may be administered to a subject by the same or by a different route.

### Use of Biomarkers to Predict Sensitivity

The present invention provides, for example, a method for evaluating sensitivity of malignant or neoplastic cells to an ERK1 or ERK2 or MEK inhibitor comprising determining if said cells comprise a V600E or V600D BRAF allele or any BRAF allele characterized by a gain-of-function phenotype (e.g., with hetero- or homozygosity for said allele); wherein said cells are determined to be sensitive if said genotype is detected.

The present invention provides embodiments wherein the cells are homozygous or heterozygous for the V600E or V600D BRAF allele. Cells with the homozygous V600E or V600D BRAF genotype are particularly sensitive to MEK or ERK1/2 inhibitors.

The present invention comprises embodiments wherein a given tumor type, e.g., as specified in Table 2 (see below), is determined to comprise at least as much (e.g., exactly or about as much) ERK or MEK inhibitor sensitivity as is observed in a cell line of that tumor type (e.g., expressed as IC50) if cells from the tumor comprise the corresponding genotype as specified in Table 2. For example, in an embodiment of the invention, a melanoma comprising a V600E homozygous genotype, as was observed in connection with the Malme 3M cell line, is determined to be sensitive to an ERK or MEK inhibitor. The present invention also comprises embodiments wherein, if a cell of a given tumor type is determined to be sensitive based on observation of a single copy of a given allele of V600D or V600E BRAF mutation, then other cells, of that tumor type, comprising a further copy of V600D or V600E mutation (e.g., homozygous V600E, homozygous V600D or heterozygous V600E/heterozygous V600D) would similarly be determined to be sensitive.

In an embodiment of the invention, a non-small cell lung cancer tumor is determined to be ERK or MEK inhibitor sensitive if it comprises one or more BRAF V600D or V600E alleles. In an embodiment of the invention, a cholangiocarcinoma tumor is determined to be MEK or ERK inhibitor sensitive if it comprises one or more V600D or V600E alleles.

The genotype can be determined using standard methods known in the art, including, e.g., methods discussed herein. For example, pyrosequencing or real-time polymerase chain reaction (RT-PCR) methods, e.g., Taqman assays, may be used for genotype detection.

Cells whose sensitivity may be evaluated using such a method may be obtained from any source. For example, cells may be obtained from a solid tumor in a subject, for example, from a biopsy or from a non-solid cancer (e.g., leukemia) by blood, serum or plasma sample. Alternatively, cells may be obtained from an in vitro source, for example, from a cell culture (e.g., from ATCC).

For example, a method for evaluating the sensitivity of a cell to an ERK1 or ERK2 or MEK inhibitor comprises (a) obtaining a sample of one or more malignant or neoplastic cells from the body of a subject (e.g., biopsy of cells from a solid tumor of a blood sample from a subject with a blood cancer); (b) determining if said malignant or neoplastic cells comprise a V600E or V600D BRAF allele or any BRAF allele characterized by a gain-of-function phenotype (e.g., the homozygous or heterozygous genotype); wherein the cells are determined to be sensitive to said inhibitor if said genotype is detected in said cells.

The present invention also provides methods for evaluating the sensitivity of a cell taken from a patient (e.g., a tumor cell) wherein steps are carried out in the absence of the patient from whom the cell was taken. For example, such a method may comprise (a) obtaining a sample of one or more malignant or neoplastic cells from the body of a subject (e.g., biopsy of cells from a solid tumor of a blood sample from a subject with a blood cancer); (b) sending the sample to a laboratory or other suitable testing facility or third party (e.g., a laboratory technician) for determining if said malignant or neoplastic cells comprise a V600E or V600D BRAF allele; and (c) determining if the cells in the sample comprise any such allele (e.g., in the absence of the patient); wherein the cells are determined to be sensitive to said inhibitor if said genotype is detected in said cells. Alternatively the present invention provides methods wherein the steps of determining sensitivity are carried out independently of any patient. For example, the present invention provides such methods comprising determining if said malignant or neoplastic cells (e.g., obtained from any source) comprise a V600E or V600D BRAF allele; wherein the cells are determined to be sensitive to said inhibitor if said genotype is detected in said cells.

The methods for evaluating sensitivity of a malignant or neoplastic cell to an ERK1/2 or MEK inhibitor may be used in any of a number of useful applications. For example, the method may aid in the selection of subjects who are candidates for receipt of an ERK1/2 or MEK inhibitor for treatment of cancer in the subject; identification of a subject with malignant or neoplastic cells which are sensitive to said inhibitor; selection of a therapy which would be appropriate or advantageous to a subject with malignant or neoplastic cells; or selection of an appropriate dosage of said inhibitor.

In an embodiment of the invention, if the malignant or neoplastic cells, in a subject, are determined to be sensitive by a method set forth herein (by identification of the V600E or V600D BRAF allele in the cell analyzed), the subject is selected for treatment with the inhibitor. In an embodiment of the invention, if the subject is selected, treatment may be commenced; wherein the subject is administered a therapeutically acceptable or effective dose of the inhibitor, optionally in association with a further chemotherapeutic (e.g., anti-cancer) agent and/or therapeutic (e.g., anti-cancer) procedure.

In an embodiment of the invention, a method for evaluating ERK1/2 or MEK inhibitor sensitivity as discussed herein may be used to identify a subject with malignant or neoplastic cells sensitive to an ERK1 or ERK2 or MEK inhibitor. In such a method, the subject is identified as having sensitive cells if the cells which are analyzed are evaluated as such by identification of the V600E or V600D BRAF allele in the cell analyzed.

The methods for evaluating malignant or neoplastic cell sensitivity to an ERK1/2 or MEK inhibitor may also be used as a basis on which to select an appropriate therapy for a subject with said cells. If the cells in the subject are determined to be sensitive to the inhibitor (by identification of the V600E or V600D BRAF allele in the cell analyzed), the inhibitor is selected as the therapy.

The present invention also provides a method for treating a tumor or cancerous condition with an ERK1 or ERK2 or MEK inhibitor comprising evaluating sensitivity of malignant or neoplastic cells, which are in said tumor or which mediate said cancerous condition, to said inhibitor and, if the cells are determined to be sensitive (by identification of the V600E or V600D BRAF allele in the cell analyzed), continuing or commencing treatment by administering, to the subject, a therapeutically effective dose of the inhibitor.

Furthermore, the present invention provides a method for selecting a dose of an ERK1 or ERK2 or MEK inhibitor to be administered, to a subject, with a tumor or cancerous condition, comprising evaluating sensitivity of malignant or neoplastic cells in the tumor or mediating the cancerous condition comprising evaluating sensitivity of the cells to the inhibitor; wherein a low dose is selected if said cells are determined to be sensitive (by identification of the V600E or V600D BRAF allele in the cell analyzed) and a high dose (e.g., upper half of the 0.04 mg/day to 4000 mg/day dose range set forth herein) is selected if said cells are determined to be less sensitive. If the cells being treated are highly sensitive to the inhibitor, less of the inhibitor (e.g., lower half of the 0.04 mg/day to 4000 mg/day dose range set forth herein) may be needed to reach the same therapeutic outcome than if the cells were insensitive or if the cells exhibited low sensitivity. The exact dosage adjustment may be made by a treating physician or clinician based on the needs of the subject time as well as the subject's particular characteristics, other medications, medical background, needs and the exigencies of the particular situation.

The present invention also provides a method of advertising an ERK1 or ERK2 or MEK inhibitor or a pharmaceutically acceptable composition thereof or a therapeutic regimen comprising administration of said inhibitor or composition comprising promoting, to a target audience, the use of the inhibitor or composition for treating a patient or patient population whose tumors or cancerous conditions are mediated by malignant or neoplastic cells that comprise a V600E or V600D BRAF allele. Advertising may take any form including, for example, print, audio, electronic or visual media.

The present invention further provides an article of manufacture comprising, packaged together, an ERK1 or ERK2 or MEK inhibitor or a pharmaceutical composition thereof comprising a pharmaceutically acceptable carrier; and a label stating that the inhibitor or pharmaceutical composition is indicated for treating patients having a tumor comprising malignant or neoplastic cells or a cancerous condition mediated by malignant or neoplastic cells that comprise a V600E or V600D BRAF allele. A method for manufacturing an ERK1 or ERK2 or MEK inhibitor or a pharmaceutical composition thereof comprising a pharmaceutically acceptable carrier is also provided, said method comprising combining, in a package, the inhibitor or composition; and a label or package insert conveying that the inhibitor or composition is indicated for treating patients having a tumor comprising malignant or neoplastic cells or a cancerous condition mediated by malignant or neoplastic cells that comprise a V600E or V600D BRAF allele. The package insert may be any acceptable medium, for example, a printed paper insert. Other relevant information may also be included in such a package insert, including, for example, information relating to pharmacokinetics, pharmacodynamics, clinical studies, efficacy parameters, indications and usage, contraindications, warnings, precautions, adverse reactions, overdosage, proper dosage and administration, how supplied, proper storage conditions, references and patent information.

The present invention further comprises kits for predicting the sensitivity of a cell taken from a patient (e.g., a tumor cell) to an ERK1 or ERK2 or MEK inhibitor comprising the inhibitor and a set of reagents for determining if the genotype of the cell comprises a homozygous or heterozygous V600E BRAF genotype or a homozygous or heterozygous V600D BRAF genotype or any BRAF genotype characterized by a gain-of-function phenotype. For example, PCR primers useful for allelic determination may be included in such a kit (e.g., primers or other reagents useful for performing the allele detection methods set forth below under Analysis and determination of V600E or V600D BRAF alleles).

As discussed above, the present methods may be used to predict sensitivity of an in vitro cell or cell line or a cell in a xenograft (e.g., human cell in a mouse model) to an ERK1 or ERK2 or MEK inhibitor. For example, the genotype of an in vitro cell or a cell to be added to a mouse xenograft model may be determined using, e.g., any of the methods discussed herein. Following genotype determination, for example, the true level of sensitivity of the cell (e.g., IC50) may be determined with respect to any ERK or MEK inhibitor including, but not limited to, any inhibitor set forth herein.

### Analysis and Determination of V600E or V600D BRAF Alleles

An aspect of the invention includes determining whether a subject's genotype includes a particular allele of BRAF (V600E or V600D). Genotype can be determined in a subject by any of the numerous methods known in the art, e.g., by allele specific hybridization, primer extension, allele specific oligonucleotide ligation, sequencing, Taqman analysis and pyrosequencing.

Allele specific hybridization, also known as ASO (allele specific oligonucleotide hybridization), relies on distinguishing between two DNA molecules differing by one base by hybridization. Sample DNA is examined by hybridization to primers which encode the polymorphism being sought and which are immobilized to a solid substrate. Sample DNA, to be evaluated for presence of the SNP, is denatured and allowed to hybridize to the oligonucleotide/solid support. The portion of the sample DNA annealed to the immobilized oligonucleotide also encodes the SNP. The sample DNA/oligonucleotide/solid support complex is optionally washed and a detectable probe is annealed to an un-annealed, adjacent portion of the sample DNA. The presence of the probe on the complex indicates the presence of the SNP in the sample DNA. Several detectable probes are known in the art. For example, Bao et al. (Nucleic Acids Res (2005) 33(2): e15) disclose use of a gold nanoparticle probe.

The primer extension method comprises amplifying the target region by PCR followed by a single base sequencing reaction using a primer that anneals one base shy of the polymorphic site. In general, primer extension is a two step process that first involves the hybridization of a probe to the bases immediately upstream of the SNP nucleotide followed by a ‘mini-sequencing’ reaction, in which DNA polymerase extends the hybridized primer by adding a base that is complementary to the SNP nucleotide. This incorporated base is detected and determines the SNP allele. Because primer extension is based on the highly accurate DNA polymerase enzyme, the method is generally very reliable. Generally, there are two main approaches which use the incorporation of either fluorescently labeled dideoxynucleotides (ddNTP) or fluorescently labeled deoxynucleotides (dNTP). When using the ddNTP primer extension method, oligonucleotide probes hybridize to the target DNA immediately upstream of SNP nucleotide, and a single, ddNTP complementary to the SNP allele is added to the 3′ end of the probe (the missing 3′-hydroxyl in dedioxynucleotide prevents further nucleotides from being added) when DNA polymerase is added. Each ddNTP is labeled with a different fluorescent signal. Thus, detection of an extension product with a particular fluorescent signal will indicate that one ddNTP or another was added to the 3′ end of the primer. This, in turn, will indicate which nucleotide is present at the position of interest.

When using the dNTP primer extension method, allele-specific probes that have 3′ bases which are complementary to each of the SNP alleles being interrogated are used. If the target DNA contains an allele complementary to the probe's 3′ base, the target DNA will completely hybridize to the probe, allowing DNA polymerase to extend from the 3′ end of the probe. Extension is detected by the incorporation of fluorescently labeled dNTPs onto the end of the probe. If the target DNA does not contain an allele complementary to the probe's 3′ base, the target DNA will produce a mismatch at the 3′ end of the probe and DNA polymerase will not be able to extend from the 3′ end of the probe.

Allele specific oligonucleotide ligation relies on the ability of DNA ligase to catalyze the ligation of the 3′ end of a DNA fragment to the 5′ end of a directly adjacent DNA fragment. This mechanism can be used to interrogate a SNP by hybridizing two probes directly over the SNP polymorphic site, whereby ligation can occur if the probes are identical to the target DNA. In the oligonucleotide ligase assay, two probes are designed; an allele-specific probe which hybridizes to the target DNA so that it's 3′ base is situated directly over the SNP nucleotide and a second probe that hybridizes upstream of the SNP polymorphic site providing a 5′ end for the ligation reaction. If the allele-specific probe matches the target DNA, it will fully hybridize to the target DNA and ligation can occur. Ligation does not generally occur in the presence of a mismatched 3′ base. Ligated or unligated products can be detected e.g., by gel electrophoresis, MALDI-TOF mass spectrometry or by capillary electrophoresis.

Sequencing is a common method for SNP detection. The most common forms of sequencing are based on primer extension using either a) dye-primers and unlabeled terminators or b) unlabeled primers and dye-terminators. The products of the reaction are then separated using electrophoresis using either capillary electrophoresis or slab gels.

Taq DNA polymerase's 5′-nuclease activity is used in the Taqman assay for SNP genotyping. The Taqman assay is performed concurrently with a PCR reaction and the results can be read in real-time as the PCR reaction proceeds. The assay requires forward and reverse PCR primers that will amplify a region that includes the SNP polymorphic site. Allele discrimination is achieved using FRET combined with one or two allele-specific probes that hybridize to the SNP polymorphic site. The probes will have a fluorophore linked to their 5′ end and a quencher molecule linked to their 3′ end. While the probe is intact, the quencher will remain in close proximity to the fluorophore, eliminating the fluorophore's signal. During the PCR amplification step, if the allele-specific probe is perfectly complementary to the SNP allele, it will bind to the target DNA strand and then get degraded by 5′-nuclease activity of the Taq polymerase as it extends the DNA from the PCR primers. The degradation of the probe results in the separation of the fluorophore from the quencher molecule, generating a detectable signal. If the allele-specific probe is not perfectly complementary, it will have lower melting temperature and not bind as efficiently. This prevents the nuclease from acting on the probe (McGuigan et al., Psychiatr. Genet. (2002) 12(3):133-136).

Cells which are, in embodiments of the present invention, evaluated for the presence of a given genotype may be obtained from a subject in any reasonable manner. Embodiments of the invention include those wherein the cells are obtained from the subject's body by surgical biopsy (e.g., endoscopic biopsy, excisional or incisional biopsy, fine needle aspiration (FNA) biopsy,). For example, a skin biopsy may be taken by surgical excision, shave biopsy or by punch biopsy of a skin sample suspected of being or known to be melanoma. Such samples may also be obtained, particularly wherein the disease involved is a non-solid tumor disease such as leukemia, by taking a blood, serum or plasma sample from said subject or by taking a sample of the patient's bone marrow (e.g., from the sternum or iliac crest hipbone). Subsequent processing of such biopsy samples for determination of genotype may be performed using methods known in the art.

Pyrosequencing may also be used to determine the presence of a V600 mutation in a cell (Spittle et al., J. Molec. Diagnostics (2007) 9(4): 464-471). Pyrosequencing is a technique well known in the art. Briefly, pyrosequencing involves sequencing a small region surrounding a chromosomal location of interest using detectably labeled nucleotides. Commercially available kits may be purchased for sequencing codon 600 of BRAF (see e.g., Pyromark™ BRAF, Biotage, AB; Isogen Life Science, Netherlands).

### EXAMPLES

The present invention is intended to exemplify the present invention and not to be a limitation thereof. Any method or composition disclosed below falls within the scope of the present invention.

### Example 1

### Identification and Evaluation of BRAF Biomarker

In this example, the BRAF V600 biomarkers were identified and determined to be an accurate predictor of the sensitivity of a cell to an ERK1 or ERK2 or MEK inhibitor.

### Results

We have determined the BRAF genotype status for 41 tumor cell lines to identify cell lines that contain wild-type BRAF, heterozygous V600E BRAF mutations, homozygous V600E BRAF mutations or heterologous V600D BRAF mutations. In addition, the cell proliferation IC50 values for an ERK1/2 kinase inhibitor (compound a) and a MEK kinase inhibitor (compound b) were also determined.

When the BRAF genotype status of a cell line was compared to the cell proliferation IC50 values for the ERK1/2 inhibitor compound a or MEK inhibitor compound b, a strong correlation was seen between the V600E BRAF homozygous cell line genotype and increased IC50 sensitivity to either the ERK1/2 kinase inhibitor compound a or MEK kinase inhibitor compound b (Table 2). Cell lines containing the homozygous V600E BRAF mutation had an increased sensitivity, as measured by relatively low cell proliferation 1050 values, to compound a or b when compared to cell lines containing either the heterozygote V600E BRAF mutation or wild type BRAF genotype (Table 2). Nine of the nine V600E BRAF homozygous genotype cell lines tested have 1050 values of <100 nM for the ERK1/2 kinase inhibitor compound a (Table 2). Eight of the nine V600E BRAF homozygous genotype cell lines tested have IC50 values of ≦10 nM for the MEK kinase inhibitor compound b (Table 2). Cell lines with either the heterozygote V600E BRAF mutation or wild type BRAF genotype were less sensitive to the ERK1/2 kinase inhibitor compound a (25 of the 32 cell lines tested had IC50 values >100 nM) or MEK kinase inhibitor compound b (23 of 32 cell lines tested had IC50s of >10 nM) (Table 2). The two cell lines containing the heterozygous V600D BRAF mutation (WM-266-4 and WM-155) had increased sensitivity to both the ERK1/2 inhibitor compound a or MEK inhibitor compound b (IC50 values of <100 nM for compound a and <10 nM for compound b) (Table 2).

### Materials and Methods

To identify single nucleotide polymorphisms within the BRAF conserved kinase domain region (Exons 11-18), or BRAF exon 15 containing the V600 amino-acid of BRAF, cell line DNA samples were sequenced from tumor cell lines listed in Table 2. The BRAF kinase domain region (Accession number NM_{—}004333) UCSC Human chromosomal region chr7:140,080,753-140,271,032 which contains BRAF Exons 11-18 were sequenced using the following PCR Assays and PCR primers.

BRAF exon base pair positions. The USCS human chromosome 7, location 7q34 positions for each BRAF exon are set forth below.

Exon 1: 140,080,753 to 140,081,038, Exon 2: 140,086,080 to 140,086,214, Exon 3: 140,095,555 to 140,095,686, Exon 4: 140,099,543 to 140,099,661, Exon 5: 140,100,455 to 140,100,501, Exon 6: 140,123,180 to 140,123,356, Exon 7: 140,124,259 to 140,124,343, Exon 8: 140,127,844 to 140,127,961, Exon 9: 140,129,289 to 140,129,425, Exon 10: 140,133,816 to 140,133,852, Exon 11: 140,140,576 to 140,140,735, Exon 12: 140,146,630 to 140,146,749, Exon 13: 140,147,680 to 140,147,828, Exon 14: 140,154,228 to 140,154,330, Exon 15: 140,155,160 to 140,155,263, Exon 16: 140,180,877 to 140,181,140, Exon 17: 140,196,379 to 140,196,480, Exon 18: 140,270,834 to 140,271,032.

The UCSC Genome Browser is developed and maintained by the Genome Bioinformatics Group, a cross-departmental team within the Center for Biomolecular Science and Engineering (CBSE) at the University of California Santa Cruz (UCSC).

BRAF PCR Assays. Several polymerase chain reactions were performed to sequence the relevant BRAF exons. These reactions are summarized below.

Designations (Assay number_Forward primer Name_Reverse Primer Name)

### Exon 11: Assay 1994_{—}4825_BRAF_R_{—}4826_BRAF_F

### Exon 12: Assay 1995_{—}4835_BRAF_R_{—}4836_BRAF_F

### Exon 13: Assay 2544_{—}21968_BRAF_R21967_BRAF_F

### Exon 14: Assay1997_{—}4841_BRAF_R_{—}4842_BRAF_F

### Exon 15: Assay 1998_{—}4827_BRAF_R_{—}4828_BRAF_F

### Exon 16: Assay 1999_{—}4845_BRAF_R_{—}4846_BRAF_F

### Exon 17: Assay 2000_{—}4843_BRAF_R_{—}4844_BRAF_F

### Exon 18: Assay 2001_{—}4833_BRAF_R_{—}4834_BRAF_F

PCR primers. The primer sets used in each polymerase chain reaction discussed above are as follows:

4825_BRAF_R;
(SEQ ID NO: 3)
CAGGAAACAGCTATGACCTTGAGGACTAGTTAACCTGGAGGA
4826_BRAF_F;
(SEQ ID NO: 4)
TGTAAAACGACGGCCAGTAGAATTTTTCTTAAGGGGATCTCTTC
4827_BRAF_R;
(SEQ ID NO: 5)
CAGGAAACAGCTATGACCCACTGATTTTTGTGAATACTGGGA
4828_BRAF_F;
(SEQ ID NO: 6)
TGTAAAACGACGGCCAGTTTAGGAAAGCATCTCACCTCATC
4833_BRAF_R;
(SEQ ID NO: 7)
CAGGAAACAGCTATGACCTCTTTAACCACACAAGTGTTCTTTG
4834_BRAF_F;
(SEQ ID NO: 8)
TGTAAAACGACGGCCAGTTTTTCCCAAGCATTTATGACAA
4835_BRAF_R;
(SEQ ID NO: 9)
CAGGAAACAGCTATGACCACTTAAAAGAATGTGGTTAAAGACAAA
4836_BRAF_F;
(SEQ ID NO: 10)
TGTAAAACGACGGCCAGTCATGGAACAAACAAGGTTGG
4841_BRAF_R;
(SEQ ID NO: 11)
CAGGAAACAGCTATGACCAGGCTGTGGTATCCTGCTCT
4842_BRAF_F;
(SEQ ID NO: 12)
TGTAAAACGACGGCCAGTGGCTTGACTGGAGTGAAAGG
4843_BRAF_R;
(SEQ ID NO: 13)
CAGGAAACAGCTATGACCCCAAAATTTCTAGGTGTGCCA
4844_BRAF_F;
(SEQ ID NO: 14)
TGTAAAACGACGGCCAGTACTCCTTTTGTGGGTTTCCC
4845_BRAF_R;
(SEQ ID NO: 15)
CAGGAAACAGCTATGACCTGCGATGGTCAAGAAATATCC
4846_BRAF_F;
(SEQ ID NO: 16)
TGTAAAACGACGGCCAGTATGGTAAAAGCATTGCTCTAGGA
21967_BRAF_F;
(SEQ ID NO: 17)
TGTAAAACGACGGCCAGTAGCTTTTTCTGACAACATTTTACCG
21968_BRAF_R;
(SEQ ID NO: 18)
CAGGAAACAGCTATGACCTGCAATCCAAAAGAATAGCAGCC

Genomic DNA isolation. DNA was obtained from tumor cell lines utilizing the Qiagen DNeasy Blood and Tissue Kit according to the manufacturer's instructions. (Qiagen; Valencia, Calif.).

Polymerase Chain Reaction. PCR primers were designed using the Primer3 software (see www.genome.wi.mit.edu/cgi-bin/primer/primer3.cgi) to amplify Exons L, M, N, O, P, Q, R of BRAF. Forward and reverse primers were 5′ tailed with universal sequencing primers (−21M13: 5′ TGTAAAACGACGGCCAGT (SEQ ID NO: 19) and M13REV: CAGGAAACAGCTATGACC (SEQ ID NO: 20), respectively). PCR reactions containing Tumor cell line genomic DNA (12 ng) was PCR amplified with either FideliTaq™ PCR Master Mix (2×) (USB Corporation; Cleveland, Ohio) or AccuPrime SuperMix II (Invitrogen; Carlsbad, Calif.) according to the manufacturer's instructions.

DNA sequencing and analysis. Following DNA amplification, PCR reactions were diluted to 20 μl in PCR buffer containing 0.25 μl of ExoSAP-IT (USB Corporation; Cleveland, Ohio) and were incubated for 15 minutes at 37° C. followed by inactivation of the enzymes at 80° C. for 15 minutes. Cycle sequencing in the forward and reverse directions was performed using an ABI PRISM BigDye terminator v3.1 Cycle Sequencing DNA Sequencing Kit (Applied Biosystems; Foster City, Calif.) according to manufacture's instructions. Briefly, 1 μl of each PCR product was used as a template and combined with 4 μl of sequencing reaction mix containing 5 pmol M13 sequencing primer (−21M13 or M13Rev), 0.5× Sequencing buffer and 0.25 μl BDTv3.1 mix. Sequencing reactions were denatured for 1 minute at 96° C. followed by 25 cycles at 96° C. for 10 seconds, 50° C. for 5 seconds and 60° C. for 4 minutes. Sequencing reactions were purified by filtration using Montage SEQ384 plates (Millipore Corp.; Bedford, Mass.), dissolved in 25 μl deionized water and resolved by capillary gel electrophoresis on an Applied Biosystems 3730XL DNA Analyzer. Chromatograms were transferred to a Unix workstation (DEC alpha, Compaq Corp), base called with Phred (version 0.990722.g), assembled with Phrap (version 3.01) scanned with Polyphred (version 3.5) {Nickerson, 1997 #33} and the results viewed with Consed (version 9.0) (Phred, Phrap and Consed available at www.genome.washington.edu, PolyPhred is available at droog.mbt.washington.edu). Analysis parameters were all maintained at the individual software's default settings.

The BRAF amino acid sequence is set forth below. The BRAF region sequenced in the studies discussed herein is set forth below in bold faced, underscored font.

(SEQ ID NO: 2)
MAALSGGGGGGAEPGQALFNGDMEPEAGAGAGAAASSAADPAIPEEVWN
IKQMIKLTQEHIEALLDKFGGEHNPPSIYLEAYEEYTSKLDALQQREQQ
LLESLGNGTDFSVSSSASMDTVTSSSSSSLSVLPSSLSVFQNPTDVARS
NPKSPQKPIVRVFLPNKQRTVVPARCGVTVRDSLKKALMMRGLIPECCA
VYRIQDGEKKPIGWDTDISWLTGEELHVEVLENVPLTTHNFVRKTFFTL
AFCDFCRKLLFQGFRCQTCGYKFHQRCSTEVPLMCVNYDQLDLLFVSKF
FEHHPIPQEEASLAETALTSGSSPSAPASDSIGPQILTSPSPSKSIPIP
QPFRPADEDHRNQFGQRDRSSSAPNVHINTIEPVNIDDLIRDQGFRGDG
GSTTGLSATPPASLPGSLTNVKALQKSPGPQRERKSSSSSEDRNRMKTL
GRRDSSDDWEIPDGQITVGQRIGSGSFGTVYKGKWHGDVAVKMLNVTAP
TPQQLQAFKNEVGVLRKTRHVNILLFMGYSTKPQLAIVTQWCEGSSLYH
HLHIIETKFEMIKLIDIARQTAQGMDYLHAKSIIHRDLKSNNIFLHEDL
TVKIGDFGLATVKSRWSGSHQFEQLSGSILWMAPEVIRMQDKNPYSFQS
DVYAFGIVLYELMTGQLPYSNINNRDQIIFMVGRGYLSPDLSKVRSNCP
KAMKRLMAECLKKKRDERPLFPQILASIELLARSLPKIHRSASEPSLNR
AGFQTEDFSLYACASPKTPIQAGGYGAFPVH

IC50 Cell proliferation measurements. Cell proliferation was assessed in the 41 tumor cell lines set forth in Table 2 after 4 days of continuous exposure to either the ERK1/2 inhibitor, compound a or the MEK inhibitor compound b (PD0325901) using Promega Cell Titer Glo reagent (Promega Corp, Madison, Wis.). Promega's CellTiter-Glo™ Luminescent Cell Viability Assay is a sensitive method for assaying cell proliferation using a stable form of luciferase to measure ATP as an indicator of viable cells. This reagent allows a homogeneous method of determining cell number by quantitation of ATP. The luciferase luminescent signal produced is proportional to the number of viable cells present in culture.

A 1000× compound dose response curve, containing 8 dilutions of each compound, was prepared on a 96-well plate. Compounds were diluted in 100% DMSO to the following (1000×) concentrations: compound b: 1000 μM, 333 μM, 111 μM, 37 μM, 12 μM, 4.1 μM, 1.4 μM and 0.5 μM; compound a: 3000 μM, 1000 μM, 333 μM, 111 μM, 37 μM, 12 μM, 4.1 μM and 1.4 μM. This compound source plate was sealed and kept frozen between uses.

Cells were grown in RPMI 1640 medium supplemented with 10% Fetal Bovine Serum, 2 mM Glutamine and non-essential amino acids or DMEM/F12 Media containing the same supplements. On day zero, cells were trypsinized, counted and plated into wells of a 96-well Wallac TC Isoplate (Perkin-Elmer Wallac, Gaithersburg, Md.) at a density of 1500-2000 cells per well in 50 μl of complete media. An additional 6-8 replicate well of the cells at the same density were plated on a separate Wallac TC Isoplate. This plate was developed on day 1 to determine the cell-growth baseline.

On day 1 of treatment, compound was diluted to 2× in media and immediately added to the cells as follows: one ml of media was added to each well of a deep-well 96-well plate and 2 μl of 1000× compound was added with mixing to make 2× compound in media. 50 μl of the media containing 2× compound was added to the cell plates in duplicate points so the final concentrations for each well were as outlined in Table 1. The plates were then returned to a 37° C. incubator and left undisturbed for 4 days. The day 1 (baseline) plates were developed by first adding 50 μl media (without compound) to bring the volume in each well to 100 μl, then adding 100 μl of Cell Titer Glo reagent (as below).

After four days of treatment, the plates were developed with Cell Titer Glo (CTG) reagent (Promega Corp.; Madison, Wis.). Reagent was reconstituted as described in the product insert. 100 μl of reagent was added to all treatment and control wells and the plates were gently shaken for 5 minutes then incubated an additional 5 minutes before reading in an Analyst AD (Molecular Devices Corporation; Sunnyvale, Calif.) using the Luminescence settings.

### Analysis

Data was analyzed in Excel using XLfit 4.2, fit model #205 (Dose Response One Site)

Fit equation: Fit=(A+((B−A)/(1+((C/x)̂D))))

### Where:

A=Baseline (day 1) CTG reading

B=4 day growth CTG reading

C=IC₅₀

D=Hill slope.

x=Log concentration of compound (nM)

The number of cell doubling from day one was also calculated—results were not used if the cell doubling was less than 2.0.

Materials used were as follows:

| | | |
|---|---|---|
| RPMI-1640 Media | Gibco (Invitrogen) | 11875 |
| DMEM/F12 Media | Gibco (Invitrogen) | |
| 200 mM L-Glutamine | Gibco (Invitrogen) | 25030 |
| MEM Non-Essential Amino Acids | Gibco (Invitrogen) | 11140 |
| Fetal Bovine Serum | Gibco (Invitrogen) | 10082-147 |
| Trypsin | Gibco (Invitrogen) | 25200 |
| Isoplate TC | Wallac | 1450-516 |
| DMSO | Sigma-Aldrich | 276855-100 |
| Cell Titer Glo Reagent | Promega | G7571 |

TABLE 1
96-well plate layout for compound dose response curves
with duplicate points.

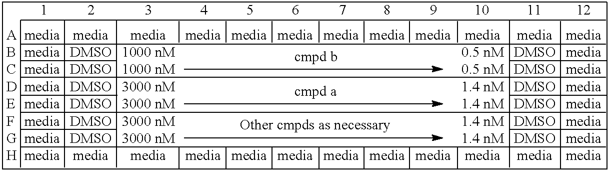

TABLE 2
Correlation of BRAF V600 biomarkers with inhibitor
sensitivity in various cell lines.

| | | ERK1/2 | MEK | |
|---|---|---|---|---|
| | | inhibitor | inhibitor | |
| | | cpd. a | cpd. b | |
| | Cell line | IC50* | IC50* | |
| Cell line | type | (nM) | (nM) | BRAF Genotype |
| Malme | Melanoma | 10 | 10 | V600E Homozygous |
| 3M | | | | |
| WM-266- | Melanoma | 20 | 10 | V600D Heterozygous |
| 4 | | | | |
| UACC- | Melanoma | 30 | 5 | V600E Homozygous |
| 62 | | | | |
| Colo-205 | Colon | 36 | 5 | V600E Homozygous |
| SK-Mel-1 | Melanoma | 37 | 3 | V600E Heterozygous |
| WiDr | Colon | 39 | 6 | V600E Heterozygous |
| M14 | Melanoma | 40 | 3 | V600E Homozygous |
| HT-29 | Colon | 50 | 20 | V600E Heterozygous |
| 8505C | Thyroid | 50 | 10 | V600E Homozygous |
| HT-144 | Melanoma | 60 | 10 | V600E Homozygous |
| WM-115 | Melanoma | 60 | 10 | V600D Heterozygous |
| SK-Mel-5 | Melanoma | 66 | 8 | V600E Heterozygous |
| A375 | Melanoma | 75 | 5 | V600E Homozygous |
| SK-Mel- | Melanoma | 85 | 10 | V600E Homozygous |
| 28 | | | | |
| H292 | Lung | 90 | 5 | V600V Wild type |
| LOX | Melanoma | 100 | 57 | V600E Homozygous |
| SK-Mel3 | Melanoma | 118 | 8 | V600E Heterozygous |
| A2058 | Melanoma | 120 | 10 | V600E Heterozygous |
| IGROV-1 | Ovarian | 146 | 39 | V600V Wild type |
| SK-Mel- | Melanoma | 150 | 18 | V600E Heterozygous |
| 31 | | | | |
| Hs695T | Melanoma | 165 | 29 | V600E Heterozygous |
| BxPc-3 | Pancreas | 184 | 119 | V600V Wild type |
| BHT 101 | Thyroid | 300 | 10 | V600E Heterozygous |
| N-87 | Gastric | 307 | 64 | V600V Wild type |
| H716 | Colon | 334 | 164 | V600V Wild type |
| RPMI- | Melanoma | 344 | 69 | V600E Heterozygous |
| 7951 | | | | |
| TT | Thyroid | 406 | 255 | V600V Wild type |
| Caki-1 | Renal | 450 | 180 | V600V Wild type |
| MB-453 | Breast | 672 | 1000 | V600E Heterozygous |
| KG-1 | Leukemia | 900 | 40 | V600V Wild type |
| Hs746T | Gastric | 125 | 7 | V600V Wild type |
| Hs294T | Melanoma | 1725 | 71 | V600E Heterozygous |
| SJCRH30 | Rhabdomyo- | 2002 | 1000 | V600V Wild type |
| | sarcoma | | | |
| ES-2 | Ovarian | 2659 | 1000 | V600E Heterozygous |
| SNU-16 | Gastric | 3000 | 1000 | V600V Wild type |
| Daudi | Leukemia | 3000 | 1000 | V600V Wild type |
| Jijoye | Leukemia | 3000 | 1000 | V600V Wild type |
| Jurkat | Leukemia | 3000 | 1000 | V600V Wild type |
| U-937 | Leukemia | 3000 | 1000 | V600V Wild type |
| A204 | Rhabdomyo- | 3000 | 1000 | V600V Wild type |
| | sarcoma | | | |
| A673 | Rhabdomyo- | 3000 | 1000 | V600E Heterozygous |
| | sarcoma | | | |

*IC50 is the concentration of the indicated compound that will cause a 50% reduction in the proliferation of the indicated cell.

As is discussed above, in an embodiment of the invention, a cell is generally considered more sensitive to an ERK1 or ERK2 inhibitor if growth inhibition is characterized by an IC50 value of about 100 nM or lower. An IC50 value of over 100 nM is generally considered resistant (i.e., less sensitive). A cell is generally considered more sensitive to a MEK inhibitor is its growth inhibition is characterized by an 1050 value of about 10 nM or lower. An IC50 value of over 10 nM is generally considered resistant (i.e., less sensitive).

The present invention is not to be limited in scope by the specific embodiments described herein. Indeed, the scope of the present invention includes embodiments specifically set forth herein and other embodiments not specifically set forth herein; the embodiments specifically set forth herein are not necessarily intended to be exhaustive. Various modifications of the invention in addition to those described herein will become apparent to those skilled in the art from the foregoing description. Such modifications are intended to fall within the scope of the appended claims.

Patents, patent applications, publications, product descriptions, and protocols are cited throughout this application, the disclosures of which are incorporated herein by reference in their entireties for all purposes.

We claim:

1. A method for evaluating sensitivity of malignant or neoplastic cells to an ERK1 or ERK2 or MEK inhibitor comprising determining if said cells are characterized by a homozygous or heterozygous V600E BRAF genotype or a homozygous or heterozygous V600D BRAF genotype or any genotype characterized by BRAF gain-of-function phenotype; wherein said cells are determined to be sensitive if said genotype is detected.

2. The method of claim 1 wherein said malignant or neoplastic cells mediate a medical condition selected from the group consisting of gastric cancer, renal cancer, non-solid cancer, rhabdomyosarcoma, cholangiocarcinoma, lung cancer, pancreatic cancer, colon cancer, myeloid leukemias, thyroid cancer, myelodysplastic syndrome, bladder carcinoma, epidermal carcinoma, melanoma, breast cancer, prostate cancer, head and neck cancers, ovarian cancer, brain cancers, cancers of mesenchymal origin, sarcomas, tetracarcinomas, neuroblastomas, kidney carcinomas, hepatomas, non-Hodgkin's lymphoma, multiple myeloma, and anaplastic thyroid carcinoma.

3. The method of claim 1 wherein said inhibitor is represented by a structural formula selected from the group consisting of:

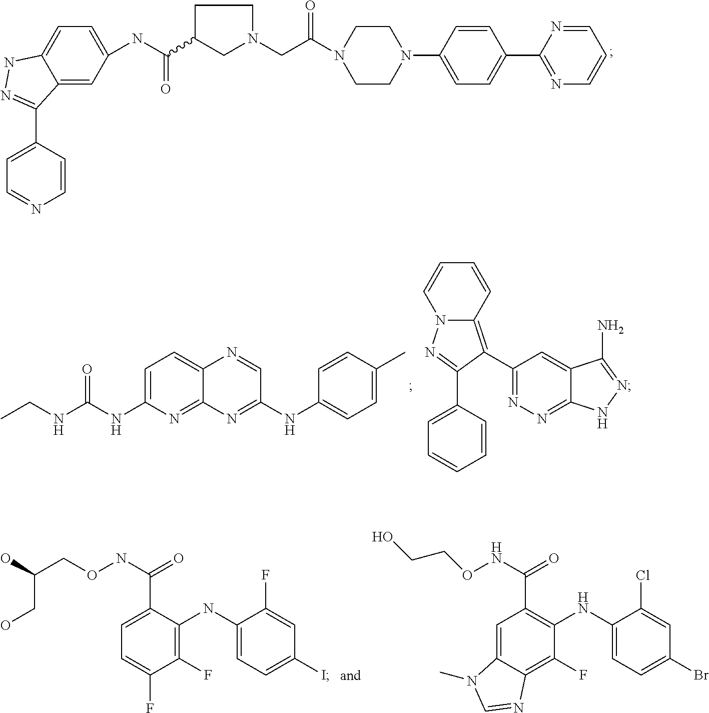

4. The method of claim 1 wherein it is determined if the cells comprise a homozygous V600E BRAF genotype.

5. The method of claim 1 wherein the cells are obtained from an in vitro source.

6. The method of claim 1 wherein the cells are obtained from an in vivo source.

7. The method of claim 1 comprising:
(a) obtaining a sample of one or more malignant or neoplastic cells from the body of a subject;
(b) determining if said malignant or neoplastic cells are characterized by a homozygous or heterozygous V600E BRAF genotype or a homozygous or heterozygous V600D BRAF genotype or any genotype of BRAF characterized by a gain-of-function phenotype; wherein the cells are determined to be sensitive to said inhibitor if said genotype is detected in said cells.

8. The method of claim 7 wherein said malignant or neoplastic cells mediate a medical condition selected from the group consisting of gastric cancer, any renal cancer, non-solid cancer, rhabdomyosarcoma, cholangiocarcinoma, lung cancer, pancreatic cancer, colon cancer, myeloid leukemias, thyroid cancer, myelodysplastic syndrome, bladder carcinoma, epidermal carcinoma, melanoma, breast cancer, prostate cancer, head and neck cancers, ovarian cancer, brain cancers, cancers of mesenchymal origin, sarcomas, tetracarcinomas, neuroblastomas, kidney carcinomas, hepatomas, non-Hodgkin's lymphoma, multiple myeloma, and anaplastic thyroid carcinoma.

9. The method of claim 7 further comprising treating the malignant or neoplastic cells in the subject by administering a therapeutically effective amount of the inhibitor, optionally in association with a therapeutically effective amount of a further chemotherapeutic agent, to said subject, if said cells are determined to be sensitive.

10. A method for selecting a subject with malignant or neoplastic cells for treatment of said cells with an ERK1 or ERK2 or MEK inhibitor comprising evaluating sensitivity of the malignant or neoplastic cells to said inhibitor by the method of claim 1; wherein said subject is selected if said cells are determined to be sensitive.

11. The method of claim 10 wherein said malignant or neoplastic cells mediate a medical condition selected from the group consisting of gastric cancer, any renal cancer, non-solid cancer, rhabdomyosarcoma, cholangiocarcinoma, lung cancer, pancreatic cancer, colon cancer, myeloid leukemias, thyroid cancer, myelodysplastic syndrome, bladder carcinoma, epidermal carcinoma, melanoma, breast cancer, prostate cancer, head and neck cancers, ovarian cancer, brain cancers, cancers of mesenchymal origin, sarcomas, tetracarcinomas, neuroblastomas, kidney carcinomas, hepatomas, non-Hodgkin's lymphoma, multiple myeloma, and anaplastic thyroid carcinoma.

12. The method of claim 10 further comprising treating the malignant or neoplastic cells in the subject by administering to the selected subject a therapeutically effective amount of the inhibitor, optionally in association with a therapeutically effective amount of a further chemotherapeutic agent.

13. A method for identifying a subject with malignant or neoplastic cells sensitive to an ERK1 or ERK2 or MEK inhibitor comprising evaluating sensitivity of the malignant or neoplastic cells to said inhibitor by the method of claim 1; wherein said subject is identified if said cells are determined to be sensitive.

14. The method of claim 13 wherein said malignant or neoplastic cells mediate a medical condition selected from the group consisting of gastric cancer, any renal cancer, non-solid cancer, rhabdomyosarcoma, cholangiocarcinoma, lung cancer, pancreatic cancer, colon cancer, myeloid leukemias, thyroid cancer, myelodysplastic syndrome, bladder carcinoma, epidermal carcinoma, melanoma, breast cancer, prostate cancer, head and neck cancers, ovarian cancer, brain cancers, cancers of mesenchymal origin, sarcomas, tetracarcinomas, neuroblastomas, kidney carcinomas, hepatomas, non-Hodgkin's lymphoma, multiple myeloma, and anaplastic thyroid carcinoma.

15. The method of claim 13 further comprising treating the malignant or neoplastic cells in the subject by administering a therapeutically effective amount of the inhibitor, optionally in association with a therapeutically effective amount of a further chemotherapeutic agent, to said subject if the subject is identified.

16. A method for treating a medical condition mediated by malignant or neoplastic cells with an ERK1 or ERK2 or MEK inhibitor comprising evaluating sensitivity of the malignant or neoplastic cells to said inhibitor by the method of claim 1 and, if said cells are determined to be sensitive, continuing or commencing treatment with said inhibitor by administering, to the subject, a therapeutically effective dose of the inhibitor.

17. The method of claim 16 wherein said medical condition is selected from the group consisting of gastric cancer, any renal cancer, non-solid cancer, rhabdomyosarcoma, cholangiocarcinoma, lung cancer, pancreatic cancer, colon cancer, myeloid leukemias, thyroid cancer, myelodysplastic syndrome, bladder carcinoma, epidermal carcinoma, melanoma, breast cancer, prostate cancer, head and neck cancers, ovarian cancer, brain cancers, cancers of mesenchymal origin, sarcomas, tetracarcinomas, neuroblastomas, kidney carcinomas, hepatomas, non-Hodgkin's lymphoma, multiple myeloma, and anaplastic thyroid carcinoma.

18. The method of claim 16 wherein said malignant or neoplastic cells are in a tumor.

19. The method of claim 16 wherein said malignant or neoplastic cells mediate a non-solid cancer.

20. A method for selecting a therapy for a subject having a medical condition mediated by malignant or neoplastic cells comprising evaluating sensitivity of the cells to an ERK1 or ERK2 or MEK inhibitor by the method of claim 1; wherein said inhibitor is selected as the therapy if said cells are determined to be sensitive to the inhibitor.

21. The method of claim 20 wherein said medical condition is selected from the group consisting of gastric cancer, any renal cancer, non-solid cancer, rhabdomyosarcoma, cholangiocarcinoma, lung cancer, pancreatic cancer, colon cancer, myeloid leukemias, thyroid cancer, myelodysplastic syndrome, bladder carcinoma, epidermal carcinoma, melanoma, breast cancer, prostate cancer, head and neck cancers, ovarian cancer, brain cancers, cancers of mesenchymal origin, sarcomas, tetracarcinomas, neuroblastomas, kidney carcinomas, hepatomas, non-Hodgkin's lymphoma, multiple myeloma, and anaplastic thyroid carcinoma.

22. The method of claim 20 further comprising treating the medical condition in the subject by administering a therapeutically effective amount of the inhibitor, optionally in association with a therapeutically effective amount of a further chemotherapeutic agent, to said subject, if said therapy is selected.

23. A method for selecting a dose of an ERK1 or ERK2 or MEK inhibitor to be administered, to a subject, having a medical condition mediated by malignant or neoplastic cells, in a treatment regimen for treating said cells, comprising evaluating sensitivity of the cells by the method of claim 1; wherein a lower dose is selected if said cells are determined to be sensitive relative to a dose selected if said cells are not determined to be sensitive.

24. The method of claim 23 wherein said medical condition is selected from the group consisting of gastric cancer, any renal cancer, non-solid cancer, rhabdomyosarcoma, cholangiocarcinoma, lung cancer, pancreatic cancer, colon cancer, myeloid leukemias, thyroid cancer, myelodysplastic syndrome, bladder carcinoma, epidermal carcinoma, melanoma, breast cancer, prostate cancer, head and neck cancers, ovarian cancer, brain cancers, cancers of mesenchymal origin, sarcomas, tetracarcinomas, neuroblastomas, kidney carcinomas, hepatomas, non-Hodgkin's lymphoma, multiple myeloma, and anaplastic thyroid carcinoma.

25. The method of claim 23 further comprising administering the selected dose of the inhibitor, optionally in association with a therapeutically effective amount of a further chemotherapeutic agent, to said subject.
